(19) United States
(12) Patent Application Publication
(10) Pub. No.: US 2022/0175754 A1
(43) Pub. Date: Jun. 9, 2022

(54) P97 INHIBITORS FOR TREATING CANCER AND NEURODEGENERATIVE DISORDERS

(71) Applicant: Lundquist Institute for Biomedical Innovation at Harbor-UCLA Medical Center, Torrance, CA (US)

(72) Inventor: Tsui-Fen Chou, Torrance, CA (US)

(21) Appl. No.: 17/481,166

(22) Filed: Sep. 21, 2021

Related U.S. Application Data
(63) Continuation of application No. 16/687,335, filed on Nov. 18, 2019, now abandoned.
(60) Provisional application No. 62/768,759, filed on Nov. 16, 2018.

(51) Int. Cl. A61K 31/4545; A61K 31/4439; A61K 31/519; A61K 31/5377; A61P 35/00; A61K 31/496; A61K 31/5513; A61K 31/541; A61K 31/444

(52) U.S. Cl. CPC A61K 31/4545; A61K 31/4439; A61K 31/519; A61K 31/5377; A61K 31/444; A61K 31/496; A61K 31/5513; A61K 31/541; A61P 35/00

(57) ABSTRACT

The present disclosure relates generally to methods for treating multisystem degenerative diseases, hematological malignancies, solid tumors, and neurodegenerative disorders.

### CROSS REFERENCE TO RELATED APPLICATIONS

This application is a continuation of U.S. application Ser. No. 16/687,335, filed Nov. 18, 2019, which claims the benefit under 35 U.S.C. § 119(e) of U.S. Provisional Application No. 62/768,759, filed Nov. 16, 2018, the contents of each of which is incorporated by reference in its entirety into the present disclosure.

### BACKGROUND

The p97 AAA (ATPase associated with diverse cellular activities), also called VCP (valosin-containing protein), is an important therapeutic target for cancer and neurodegenerative diseases. p97 forms a hexamer composed of two AAA domains (D1 and D2) that form two stacked rings, and an N-terminal domain that binds numerous cofactor proteins. The interplay between the three domains in p97 is complex, and a deeper biochemical understanding is needed in order to design selective p97 inhibitors as therapeutic agents.

The highly conserved and abundant p97 protein belongs to the type II AAA ATPase enzyme family and converts chemical energy from ATP hydrolysis into mechanical energy to unfold proteins or disassemble protein complexes. p97 plays a critical role in cellular processes such as Golgi membrane reassembly, membrane transport, degradation of proteins by the ubiquitin—proteasome system (UPS), regulation of myofibril assembly, cell division, and protein aggregation. Because p97 functions in many protein homeostatic regulatory processes, it is a potential therapeutic target for cancer and neurodegenerative diseases.

### SUMMARY

The present disclosure, in one embodiment, provides methods for treating multisystem degenerative diseases, hematological malignancies, solid tumors, and neurodegenerative disorders.

### BRIEF DESCRIPTION OF THE FIGURES

FIGS. 1A and 1B show the efficacy of compounds described herein against p97 mutations in vitro and in cancer cells. Panel A shows anti-mutant p97 ATPase activity of Compound 119 and panel B shows anti-resistant cancer activity of Compound 119.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

Generally, reference to a certain element such as hydrogen or H is meant to include all isotopes of that element. For example, if a group is defined to include hydrogen or H, it also includes deuterium and tritium. Hence, isotopically labeled compounds are within the scope of the invention. In some embodiments, one or more of the H in Formulae (I) or (I′) or (II) is replaced with a deuterium.

In general, “substituted” refers to an organic group (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. The present disclosure is understood to include embodiments where, for instance a “substituted alkyl” optionally contains one or more alkene and/or alkyne. A substituted group will be substituted with one or more substituents, unless otherwise specified.

In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; aryl groups; heteroaryl groups; cycloalkyl groups; heterocyclyl groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocycle and heteroaryl groups also include rings and fused ring systems in which a bond to a hydrogen atom is replaced with a bond to a carbon atom. Therefore, substituted cycloalkyl, aryl, heterocycle and heteroaryl groups may also be substituted with substituted or unsubstituted alkyl, alkenyl, and alkynyl groups as defined below.

Alkyl groups include straight chain and branched alkyl groups having from 1 to about 20 carbon atoms, and typically from 1 to 12 carbons or, in some embodiments, from 1 to 8, 1 to 6, or 1 to 4 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above. As stated above, the present disclosure is understood to include embodiments where, for instance a “substituted alkyl” optionally contains one or more alkene and/or alkyne.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include mono-, bicyclic and polycyclic ring systems, such as, for example bridged cycloalkyl groups as described below, and fused rings, such as, but not limited to, decalinyl, and the like. In some embodiments, polycyclic cycloalkyl groups have three rings. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-, 2,5- or 2,6-di-substituted cyclohexyl groups, which may be substituted with substituents such as those listed above. In some embodiments, a cycloalkyl group has one or more alkene bonds, but is not aromatic.

Bridged cycloalkyl groups are cycloalkyl groups in which two or more hydrogen atoms are replaced by an alkylene bridge, wherein the bridge can contain 2 to 6 carbon atoms if two hydrogen atoms are located on the same carbon atom, or 1 to 5 carbon atoms, if the two hydrogen atoms are located on adjacent carbon atoms, or 2 to 4 carbon atoms if the two hydrogen atoms are located on carbon atoms separated by 1 or 2 carbon atoms. Bridged cycloalkyl groups can be bicyclic, such as, for example bicyclo[2.1.1]hexane, or tricyclic, such as, for example, adamantyl. Representative bridged cycloalkyl groups include bicyclo[2.1.1]hexyl, bicyclo[2.2.1]heptyl, bicyclo[3.2.1]octyl, bicyclo[2.2.2]octyl, bicyclo[3.2.2]nonyl, bicyclo[3.3.1]nonyl, bicyclo[3.3.2]decanyl, adamantyl, noradamantyl, bornyl, or norbornyl groups. Substituted bridged cycloalkyl groups may be substituted one or more times with non-hydrogen and non-carbon groups as defined above. Representative substituted bridged cycloalkyl groups may be mono-substituted or substituted more than once, such as, but not limited to, mono-, di- or tri-substituted adamantyl groups, which may be substituted with substituents such as those listed above.

Aryl groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups.

Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Heterocycle groups include aromatic (also referred to as heteroaryl) and non-aromatic ring compounds containing 3 or more ring members, of which one or more is a heteroatom such as, but not limited to, N, O, S or B. In some embodiments, heterocycle groups include 3 to 20 ring members, whereas other such groups have 3 to 6, 3 to 10, 3 to 12, or 3 to 15 ring members. Heterocycle groups encompass unsaturated, partially saturated and saturated ring systems, such as, for example, imidazolyl, imidazolinyl and imidazolidinyl groups. The phrase “heterocycle group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocycle groups that have other groups, such as alkyl, oxo or halo groups, bonded to one of the ring members. Rather, these are referred to as “substituted heterocycle groups”. Heterocycle groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocycle groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, S or B. Heteroaryl groups include, but are not limited to, groups such as pyrrolyl, pyrazolyl, triazolyl, tetrazolyl, oxazolyl, isoxazolyl, thiazolyl, pyridyl, pyridazinyl, pyrimidinyl, pyrazinyl, thiophenyl, benzothiophenyl, furanyl, benzofuranyl, indolyl, azaindolyl (pyrrolopyridyl), indazolyl, benzimidazolyl, imidazopyridyl (azabenzimidazolyl), pyrazolopyridyl, triazolopyridyl, benzotriazolyl, benzoxazolyl, benzothiazolyl, benzothiadiazolyl, imidazopyridyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The term “amine” (or “amino”) as used herein refers to —NHR* and —NR*R* groups, wherein R* are independently hydrogen, or a substituted or unsubstituted alkyl, alkenyl, alkynyl, cycloalkyl, aryl, aralkyl, heterocyclylalkyl or heterocycle group as defined herein. In some embodiments, the amine is NH₂, methylamino, dimethylamino, ethylamino, diethylamino, propylamino, isopropylamino, phenylamino, or benzylamino.

The term “amide” refers to a —NR*R*C(O)— group wherein R* each independently refer to a hydrogen, (C₁-C₈)alkyl, or (C₃-C₆)aryl.

Those of skill in the art will appreciate that compounds of the invention may exhibit the phenomena of tautomerism, conformational isomerism, geometric isomerism and/or optical isomerism. As the formula drawings within the specification and claims can represent only one of the possible tautomeric, conformational isomeric, optical isomeric or geometric isomeric forms, it should be understood that the invention encompasses any tautomeric, conformational isomeric, optical isomeric and/or geometric isomeric forms of the compounds having one or more of the utilities described herein, as well as mixtures of these various different forms.

As readily understood by one skilled in the art, a wide variety of functional groups and other structures may exhibit tautomerism, and all tautomers of compounds as described herein are within the scope of the present invention.

Stereoisomers of compounds, also known as “optical isomers,” include all chiral, diastereomeric, and racemic forms of a structure, unless the specific stereochemistry is expressly indicated. Thus, compounds used in the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention.

By “pharmaceutically acceptable” is meant a material that is not biologically or otherwise undesirable, e.g., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. and Drug administration.

By “patient” is meant any animal for which treatment is desirable. Patients may be mammals, and typically, as used herein, a patient is a human individual.

The term “pharmaceutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate, (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

The term “solvates” is used in its broadest sense. For example, the term solvates includes hydrates formed when a compound of the present invention contains one or more bound water molecules.

Certain compounds within the scope of the disclosure are derivatives referred to as prodrugs. The expression “prodrug” denotes a derivative of a known direct acting drug, e.g. esters and amides, which derivative has enhanced delivery characteristics and therapeutic value as compared to the drug, and is transformed into the active drug by an enzymatic or chemical process; see Notari, R. E., “Theory and Practice of Prodrug Kinetics,” Methods in Enzymology 112: 309-23 (1985); Bodor, N., “Novel Approaches in Prodrug Design,” Drugs of the Future, 6: 165-82 (1981); and Bundgaard, H., “Design of Prodrugs: Bioreversible—Derivatives for Various Functional Groups and Chemical Entities,” in DESIGN OF PRODRUGS (H. Bundgaard, ed.), Elsevier (1985), Goodman and Gilmans, THE PHARMACOLOGICAL BASIS OF THERAPEUTICS, 8th ed., McGraw-Hill (1992). In some embodiments, the “prodrug” is a compound that generally converts to an active compound of the present disclosure within a physiological environment (e.g., stomach, colon, blood). Pro-drugs include esters, carbonates, carbamates, oximes of active alcohols (and/or acids for esters), amides, carbamates, ureas, oximes, Mannich bases, imines of amines (and/or acids for amides), carbondithianes of active thiols, conjugates of reactive species such as a,b-unsaturated carbonyl derivatives. The selection and synthesis of prodrugs include strategies such as those in: Karaman, R., “Prodrugs design based on inter- and intramolecular chemical processes,” Chem. Biol. Drug Des., 82: 643-668 (2013); Huttunen et al., “Prodrugs—from serendipity to rational design,” Pharmacol. Rev., 63, 750-771 (2011); Blencowe et al., “Self-immolative linkers in polymeric delivery systems,” Polym. Chem., 2: 773-790 (2011); Arpicco et al., “Anticancer prodrugs: An overview of major strategies and recent developments,” Curr. Top. Med. Chem. (Sharjah, United Arab Emirates), 11: 2346-2381 (2011); Tietze et al., “Antibody-directed enzyme prodrug therapy: A promising approach for a selective treatment of cancer based on prodrugs and monoclonal antibodies” Chem. Biol. Drug Des., 74: 205-211 (2009); Simplicio et al., “Prodrugs for amines,” Molecules, 13: 519-547 (2008); Rautio et al., “Prodrugs: Design and clinical applications,” Nat. Rev. Drug Discovery, 7: 255-270 (2008); Lee et al., “Pro-drug and Antedrug: Two Diametrical Approaches in Designing Safer Drugs,” Arch. Pharm. Res., 25: 111-136 (2002); and Lee, Chem. Biol. Drug Des., 82: 643-668 (2013). In some embodiments, the prodrug is a dimer. Two non-limiting examples are:

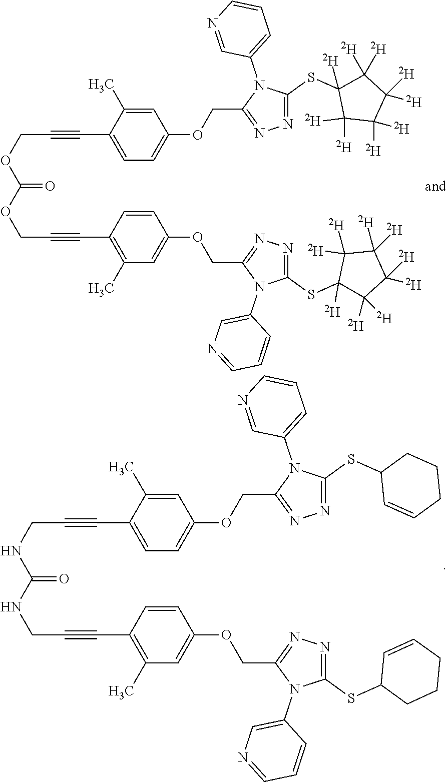

Similar analogs may be prepared based on any compound of Table III, and are included in the present technology.

Generally, reference to a certain moiety capable of being protected (such as hydroxy, amine, carbonyl, etc.) includes the protected groups in some embodiments of the disclosure. For example, in some embodiments, an —OH moiety as included herein also includes —OP, where P is a protecting group. Protecting groups, as referred to herein may be selected by one of ordinary skill in the art, and include the groups and strategies set forth in the art, for example, those such as described in R. Larock, Comprehensive Organic Transformations, VCH Publishers (1989); T. W. Greene and P. G. M. Wuts, Greene's protective groups in organic synthesis, John Wiley & Sons (2006); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### Compounds

Provided herein are compounds that having the following core structure:

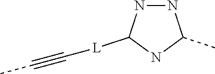

wherein L is a linking group, and the dashed lines are further functionalizations. The linking group and the further functionalizations are described in greater detail herein.

In some embodiments, compounds of the present disclosure include those represented by the following Formula (I):

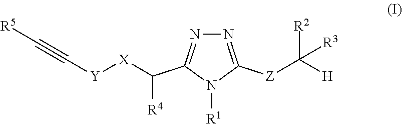

or a pharmaceutically acceptable salt, solvate or prodrug thereof, wherein:
Z is O, SO₀₋₂, NR, C(R⁷)₂, alkenyl or alkynyl;
R² and R³ are independently an optionally substituted C₁₋₉ cyclic, C₃₋₉ heterocyclic, or halogen, or R² and R³ together form an optionally substituted C₃₋₉ cyclic or 3- to 9-membered heterocyclic ring;
R¹ is optionally substituted phenyl or an optionally substituted 5- or 6-membered heteroaryl;
R⁴ is H, C(R⁷)₂, aryl, or heteroaryl;

### X is O or SO₀₋₂;

Y is an optionally substituted alkyl, alkenyl, aryl, heteroaryl, cycloalkyl, or heterocycle;
R⁵ is H, nitrile, an optionally substituted aryl, an optionally substituted heterocycle, optionally substituted C₁-C₆ alkyl, optionally substituted C₃-C₉ cycloalkyl, or

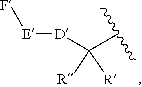

where
R′ and R″ are each independently selected from the group consisting of H, optionally substituted alkyl, —OR, halogen (e.g., F, Cl, Br, I), —N(R)₂, and optionally substituted cycloalkyl or heterocycle;
or R′ and R″ may together form a 3- to 6-membered cycloalkyl or heterocycle that is optionally substituted;
D′ is selected from the group consisting of —O—, —NR—, —OCONR—, —NRSO₂—, —NRCO—, —NRSO₂NR—, —NRCOO—, —NRCONR—, and —NRC(NR)NR—;
E′ is selected from a bond or an optionally substituted C₁-C₆ alkyl and cycloalkyl; and
F′ is selected from the group consisting of H, an optionally substituted cycloalkyl, an optionally substituted heterocycle, an optionally substituted aryl, and an optionally substituted heteroaryl;
R is selected from the group consisting of H, optionally substituted alkyl, and optionally substituted cycloalkyl; and
R⁷ is selected from the group consisting of H, halogen, optionally substituted alkyl, optionally substituted cycloalkyl, —OR, and —N(R)₂.

In Formula (I), X is one of the following moieties: O, S, SO, or SO₂.

In Formula (I), Y is an optionally substituted alkyl, alkenyl, aryl, heteroaryl, cycloalkyl, or heterocycle. In some embodiments, Y is selected from the group consisting of an optionally substituted alkyl, an optionally substituted aryl, an optionally substituted heteroaryl, an optionally substituted C₃-C₁₀ cycloalkyl, and an optionally substituted heterocycle (containing one or more O, S, SO, SO₂, B, N, or NR). In some embodiments, Y is selected from the group consisting of:

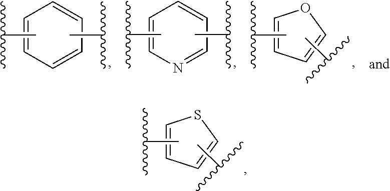

each of which may be optionally substituted. In a preferred embodiment, Y is

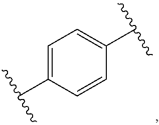 [SMILES: *c1ccc(*)cc1]

which may be optionally substituted, for example the phenylene may be substituted with alkyl or cycloalkyl, halogen, —NR₂, —SF₅ and —OR. In some embodiments, the phenylene is substituted by methyl or perfluoromethyl. In some embodiments, the substitution is at a position ortho to the alkyne. Other examples within these embodiments, include phenylene moieties substituted by one or more fluoro moiety. For example:

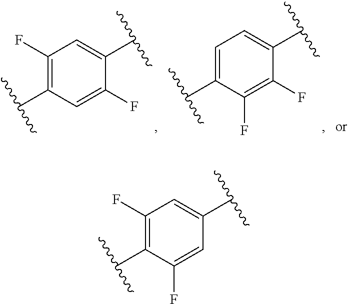 [SMILES: *c1cc(F)c(*)cc1F ; *c1ccc(*)c(F)c1F ; *c1cc(F)c(*)c(F)c1]

and further substituted variants thereof, e.g.,

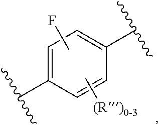

where R′″ is a halogen (e.g., F, Cl, or Br), nitrile, a C₁-C₆ alkyl, or O—C₁-C₆ alkyl.

In some embodiments, the phenylene is di-substituted, for example,

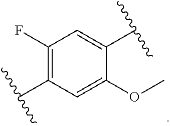 [SMILES: *c1cc(OC)c(*)cc1F]

In some embodiments, Y is phenylene optionally substituted with C₁-C₆ alkyl, C₁-C₆ haloalkyl, halogen, C₁-C₆ alkoxy, nitrile, or a combination of two or more thereof.

In some embodiments, Y is cyclobutene, propellane, cubane or

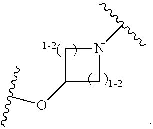

In some embodiments, Y is cyclobutene, propellane (such as, but not limited to, [1.1.1.0^{1′3}] propellane), cubane or

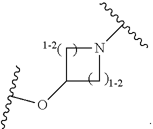

In Formula (I), Z is O, SO₀₋₂, NR, C(R⁷)₂, alkenyl or alkynyl. In some preferred embodiments, Z is selected from O, S or C(R⁷)₂, where R⁷ is independently H, halogen, optionally substituted alkyl, optionally substituted cycloalkyl, or —N(R)₂. In some embodiments, R or R⁷ is preferably H. In some embodiments, Z is S.

In Formula (I), R⁵ is H, nitrile, an optionally substituted aryl, an optionally substituted heterocycle, optionally substituted C₁-C₆ alkyl, optionally substituted C₃-C₉ cycloalkyl, or

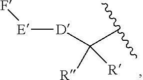

where R′ and R″ are each independently selected from the group consisting of H, optionally substituted alkyl, —OR, halogen (e.g., F, Cl, Br, or I), —N(R)₂, and optionally substituted cycloalkyl or heterocycle;
or R′ and R″ may together form a 3- to 6-membered cycloalkyl or heterocycle that is optionally substituted;
D′ is selected from the group consisting of —O—, —NR—, —OCONR—, —NRSO₂—, —NRCO—, —NRSO₂NR—, —NRCOO—, —NRCONR—, and —NRC(NR)NR—;
E′ is selected from the group consisting of a bond, an optionally substituted C₁-C₆ alkyl and cycloalkyl; and
F′ is selected from the group consisting of H, an optionally substituted cycloalkyl, an optionally substituted heterocycle, an optionally substituted aryl, and an optionally substituted heteroaryl.

When R⁵ is an optionally substituted aryl, the moiety may be, e.g., a phenyl that is unsubstituted or substituted. In some embodiments, the phenyl may be substituted with one or more of alkyl, —OR, —CN, —CO₂R, halogen, —SR, —SOR, —SO₂R, —SF₅ and —NR₂.

When R⁵ is an optionally substituted heterocycle, the moiety may be, e.g., morpholine or pyridine, optionally substituted with one or more alkyl, —OR, —CN, —CO₂R, halogen, —SR, —SOR, —SO₂R, —SF₅ or —NR₂.

In some embodiments, R⁵ is C₁-C₆ alkyl optional substituted with alkoxy, hydroxy, amino, heterocyclyl, cycloalkyl, aryl, heteroaryl, or a combination of two or more thereof. In some embodiments, R⁵ is H.

In Formula (I), when R⁵ is:

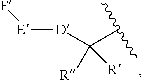

D′, E′ and F′ may be defined as follows:

D′ may be selected from the group consisting of —O—, —NR—, —OCONR—, —NRSO₂—, —NRCO—, —NRSO₂NR—, —NRCOO—, —NRCONR—, and —NRC(NR)NR—;

E′ may be selected from a bond or an optionally substituted C₁-C₆ alkyl and cycloalkyl; and
F′ may be selected from the group consisting of H, halogen, an optionally substituted cycloalkyl, an optionally substituted heterocycle, an optionally substituted aryl, and an optionally substituted heteroaryl.

D′ is —O—, —NR—, —OCONR—, —NRSO₂—, —NRCO—, —NRSO₂NR—, —NRCOO—, —NRCONR—, or —NRC(NR)NR—, where R is defined previously. In some embodiments, D′ may be selected from the group consisting of O, NH, OCONH, NHSO₂, NHCO, NHSO₂NH, NHCOO, and NHCONH.

E′ may be a bond or an optionally substituted C₁-C₆ alkyl or cycloalkyl. In some embodiments, E′ is an optionally substituted C₁-C₆ alkyl. In some embodiments, E′ is an optionally substituted C₁-C₆ alkyl and F′ is H. In other embodiments, E′ is an optionally substituted C₁-C₆ alkyl that is substituted by a C₃-C₇ spirocycle. In some embodiments, E′ may be an optionally substituted C₃-C₆ cycloalkyl, (e.g., optionally substituted with alkyl, halogen, or —OR). In some embodiments the cycloalkyl is specifically selected from, e.g., one of the following: cyclopropyl, cyclobutyl, cyclopentyl, and cyclohexyl, each of which may be optionally substituted.

F′ may be selected from the group consisting of H, an optionally substituted cycloalkyl, an optionally substituted heterocycle, an optionally substituted aryl, and an optionally substituted heteroaryl. In some embodiments, F′ is an optionally substituted cycloalkyl, an optionally substituted heterocycle, an optionally substituted aryl, and an optionally substituted heteroaryl, each of which is described below in further detail. In some embodiments, F′ is substituted with one or more alkyl, perfluoroalkyl (e.g., CF₃), —OR, —CN, —CO₂R, halogen, —SR, —SOR, —SO₂R, —SF₅ or —NR₂. In other embodiments, F′ is not substituted.

In some embodiments, F′ is an optionally substituted cycloalkyl. For example, F′ may be a C₃-C₆ cycloalkyl, optionally substituted with alkyl, halogen, or —OR. In some embodiments the cycloalkyl is specifically selected from, e.g., one of the following: cyclopropyl, cyclobutyl, cyclopentyl, and cyclohexyl, each of which may be optionally substituted. In some embodiments the cycloalkyl is specifically selected from, e.g., one of the following: cyclopropyl, cyclobutyl, cyclopentyl, cyclohexyl, and propellane, each of which may be optionally substituted. In some embodiments, E′ is a bond and F′ is an optionally substituted cycloalkyl. In some embodiments, E′ is optionally substituted C₁-C₆ alkyl and F′ is an optionally substituted cycloalkyl.

In some embodiments, F′ is an optionally substituted heterocycle. For example, F′ may be a 4-6 membered heterocycle, optionally substituted with alkyl, halogen, or OR. In some embodiments, the heterocycle contains one or more heteroatom selected from: O, S, SO, SO₂, B, N, and NR. Particular embodiments include, e.g., morpholine, azetidine, alkyl-piperidine, alkyl-piperazine, alkyl-diazepane, thiomorpholine 1,1-dioxide, isoindoline-1,3-dione, tetrahydropyran, and pyrrolidone. In some embodiments, E′ is a bond and F′ is an optionally substituted heterocycle. In some embodiments, E′ is optionally substituted C₁-C₆ alkyl and F′ is an optionally substituted heterocycle.

In some embodiments, F′ is an optionally substituted aryl. For example, F′ may be a C₆-C₁₀ aryl, e.g., a phenyl, that is optionally substituted, e.g., with one or more of alkyl, perfluoroalkyl (e.g., CF₃), —OR, —CN, —CO₂R, halogen, —SR, —SOR, —SO₂R, —SF₅ or —NR₂. In some embodiments, E′ is a bond and F′ is an optionally substituted aryl. In some embodiments, E′ is optionally substituted C₁-C₆ alkyl and F′ is an optionally substituted aryl.

In some embodiments, F′ is an optionally substituted heteroaryl. For example, F′ may be a 5-6 membered heteroaryl, optionally substituted with alkyl, halogen, or OR. In some embodiments, the heteroaryl contains one or more heteroatom selected from the group consisting of O, S, SO, SO₂, B, N, and NR. Particular embodiments include, e.g., alkyl-triazole, tetrazole, imidazole, and isoxazole. In some embodiments, E′ is a bond and F′ is an optionally substituted heteroaryl. In some embodiments, E′ is optionally substituted C₁-C₆ alkyl and F′ is an optionally substituted heteroaryl.

In Formula (I), when R⁵ is:

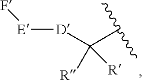

R′ and R″ may each independently selected from H, optionally substituted alkyl, —OR, halogen (e.g., F, Cl, Br, I), —N(R)₂, and optionally substituted cycloalkyl or heterocycle. In some embodiments, R′ and R″ are each H. In other embodiments, at least one of R′ and R″ is optionally substituted alkyl and any remaining R′ or R″ is H. In some embodiments, the optionally substituted alkyl is a C₁-C₆ alkyl or perfluoroalkyl. In some embodiments, the optionally substituted alkyl is methyl. In some embodiments, R′ and R″ are each independently optionally substituted C₁-C₆ alkyl.

Alternatively, R′ and R″ together may form a 3- to 6-membered cycloalkyl or heterocycle that is optionally substituted. In some embodiments, the 3- to 6-membered cycloalkyl or heterocycle is substituted by one or more R⁷, as defined previously. In additional embodiments, R′ and R″ form a cyclopropyl, cyclobutyl, cyclopentyl, or cyclohexyl. In other embodiments, R′ and R″ form an oxetane. In some embodiments, R′ and R″ form an optionally substituted azetidine, oxetane, pyrrolidone or piperidine. In some embodiments, the nitrogen of the azetidine, pyrrolidone or piperidine may be substituted with R, SO₂R, COR, SO₂NR₂, CONR₂ and COOR. In some embodiments, when R′ and R″ form a ring, e.g., an optionally substituted cyclopropyl, cyclobutyl, cyclopentyl, or cyclohexyl, azetidine, oxetane, pyrrolidone or piperidine, then D′-E′-F′ together form an —OH (i.e., D′ is —O—, E′ is a bond and F′ is H).

In Formula (I), R¹ is an optionally substituted phenyl or an optionally substituted 5- or 6-membered heteroaryl. For example, in some embodiments, R¹ is optionally substituted phenyl or optionally substituted pyridine. In some embodiments, R¹ is an unsubstituted phenyl or an unsubstituted pyridine. In some embodiments, R¹ is a pyridine. In some embodiments the pyridine is attached at the 2 position, the 3 position or the 4 position, preferably the 3 position.

In Formula (I), R² and R³ are independently an optionally substituted C₁₋₉ cyclic, C₃-9 heterocyclic, or halogen, or R² and R³ together form an optionally substituted C₃₋₉ cyclic or 3- to 9-membered heterocyclic ring. In some embodiments, R² is a C₁-C₆ alkyl, a C₃-C₁₀ cycloalkyl (including cycloalkenyl), or a heterocycle, each of which maybe optionally substituted.

In some embodiments, R² and R³ together form an optionally substituted C₃₋₉ cyclic or 3- to 9-membered heterocyclic ring, for example, a C₃-C₁₀ cycloalkyl (including cycloalkenyl), or a heterocycle, each of which maybe optionally substituted. Exemplary individual embodiments when R² and R³ together form an optionally substituted C₃₋₉ cyclic or 3- to 9-membered heterocyclic ring, include cyclopropyl, cyclobutyl, cyclopentyl, cyclohexyl, cyclopentene, and cyclohexene, each of which may be optionally substituted, for example, by one or more deuterium or fluorine moiety. Other embodiments include bicyclic structures, such as:

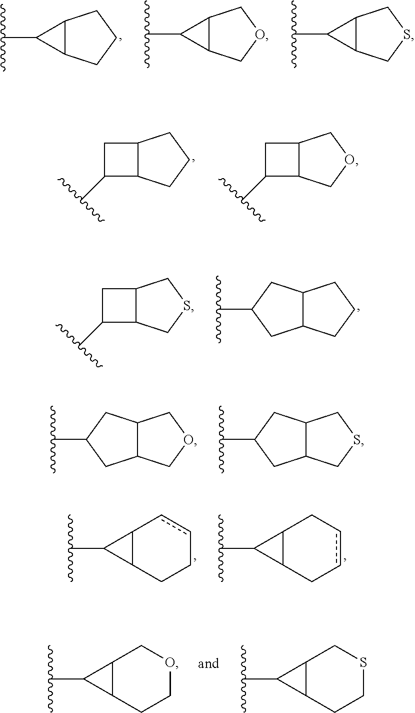

where one or more of the dashed bonds may optionally be a double bond and [symbol] indicates attachment to Z.

In some embodiments, one or more hydrogens in R² or R³, or in both R² and R³ are replaced with deuterium. In some embodiments, R² and R³ form a perdeuterated cycloalkyl ring.

In some embodiments, R² and R³ form a perdeuterated cyclopentane

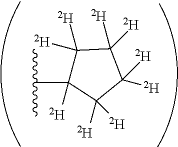

and [symbol] indicates attachment to Z. In some embodiments,

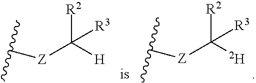

In some embodiments, compounds of the present disclosure include those represented by the following Formula (I′):

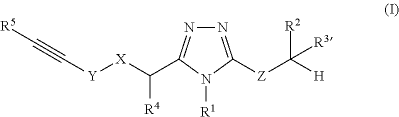

or a pharmaceutically acceptable salt, solvate or prodrug thereof, wherein:
Z is O, SO₀₋₂, NR, C(R⁷)₂, alkenyl or alkynyl;
R² and R³ are independently optionally substituted C₁₋₉ cyclic, optionally substituted C₃₋₉ heterocyclic, or halogen, or R² and R³ together form an optionally substituted C₃₋₉ cyclic or optionally substituted 3- to 9-membered heterocyclic ring;
R¹ is optionally substituted phenyl or optionally substituted 5- or 6-membered heteroaryl;
R⁴ is H, C(R⁷)₂, aryl, or heteroaryl;

### X is O or SO₀₋₂;

Y is an optionally substituted alkyl, optionally substituted alkenyl, optionally substituted aryl, optionally substituted heteroaryl, optionally substituted cycloalkyl, or optionally substituted non-aromatic heterocycle;
R⁵ is H, nitrile, an optionally substituted aryl, an optionally substituted heterocycle, optionally substituted C₁-C₆ alkyl, optionally substituted C₃-C₉ cycloalkyl, or

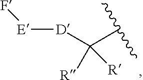

where
R′ and R″ are each independently selected from the group consisting of H, optionally substituted alkyl, —OR, halogen (e.g., F, Cl, Br, I), —N(R)₂, optionally substituted cycloalkyl, and optionally substituted heterocycle;
or R′ and R″ may together form an optionally substituted 3- to 6-membered cycloalkyl or optionally substituted 3- to 6-membered heterocycle;
D′ is selected from the group consisting of —O—, —NR—, —OCONR—, —OCO—, —NRSO₂—, —NRCO—, —NRSO₂NR—, —NRCOO—, —NRCONR—, and —NRC(NR)NR—;
E′ is selected from the group consisting of a bond, an optionally substituted C₁-C₆ alkyl, optionally substituted C₁-C₆ alkenyl, and optionally substituted cycloalkyl; and
F′ is selected from the group consisting of H, an optionally substituted cycloalkyl, an optionally substituted non-aromatic heterocycle, an optionally substituted aryl, and an optionally substituted heteroaryl;
R is independently selected from the group consisting of H, optionally substituted alkyl, and optionally substituted cycloalkyl; and
R⁷ is selected from the group consisting of H, halogen, optionally substituted alkyl, optionally substituted cycloalkyl, —OR, and —N(R)₂.

In some embodiments of a compound of Formula (I′), X is one of the following moieties: O, S, SO, or SO₂.

In some embodiments of a compound of Formula (I′), Y is selected from the group consisting of an optionally substituted alkyl, an optionally substituted alkenyl, an optionally substituted aryl, an optionally substituted heteroaryl, an optionally substituted C₃-C₁₀ cycloalkyl, and an optionally substituted non-aromatic heterocycle (containing one or more O, S, SO, SO₂, B, N, or NR). In some embodiments, Y is selected from:

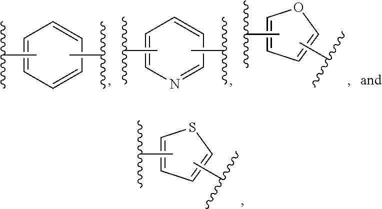

each of which may be optionally substituted.

In some embodiments, Y is

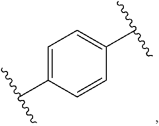 [SMILES: *c1ccc(*)cc1]

which may be optionally substituted, for example the phenylene may be substituted alkyl or cycloalkyl, halogen, —NR₂, —SF₅ and —OR. In some embodiments, the phenylene is substituted by methyl or perfluoromethyl. In some embodiments, the substitution is at a position ortho to the alkyne. Other examples within these embodiments, include phenylene moieties substituted by one or more fluoro moiety. For example, the phenylene moieties may be

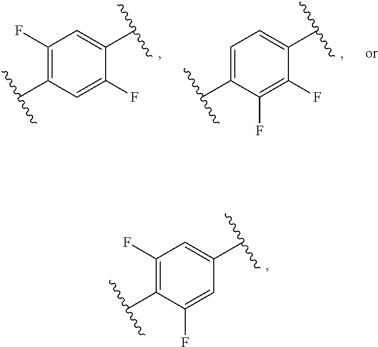 [SMILES: *c1cc(F)c(*)cc1F ; *c1ccc(*)c(F)c1F ; *c1cc(F)c(*)c(F)c1]

or further substituted variants thereof, e.g.,

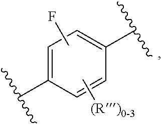

where R′″ is a halogen (e.g., F, Cl, or Br), nitrile, a C₁-C₆ alkyl, or O—C₁-C₆ alkyl. In some embodiments, the phenylene is di-substituted, for example

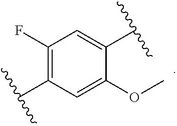 [SMILES: *c1cc(OC)c(*)cc1F]

In some embodiments, Y is phenylene optionally substituted with C₁-C₆ alkyl, C₁-C₆ haloalkyl, halogen, C₁-C₆ alkoxy, nitrile, or a combination of two or more thereof.

In some embodiments, Y is cyclobutene, propellane (such as, but not limited to, [1.1.1.0¹³] propellane), cubane or

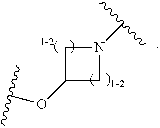

In Formula (I′), Z is O, SO₀₋₂, NR, C(R⁷)₂, alkenyl or alkynyl. In some embodiments, Z is selected from O, S or C(R⁷)₂, where R⁷ is independently H, halogen, optionally substituted alkyl, optionally substituted cycloalkyl, or —N(R)₂. In some embodiments, R or R⁷ is H. In some embodiments, Z is S.

In Formula (I′), R⁵ is H, nitrile, an optionally substituted aryl, an optionally substituted heterocycle, optionally substituted C₁-C₆ alkyl, optionally substituted C₃-C₉ cycloalkyl, or

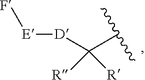

where R′ and R″ are each independently selected from the group consisting of H, optionally substituted alkyl, —OR, halogen (e.g., F, Cl, Br, or I), —N(R)₂, optionally substituted cycloalkyl, and optionally substituted heterocycle;
or R′ and R″ may together form an optionally substituted 3- to 6-membered cycloalkyl or optionally substituted 3- to 6-membered heterocycle;
D′ is selected from the group consisting of —O—, —NR—, —OCONR—, —OCO—, —NRSO₂—, —NRCO—, —NRSO₂NR—, —NRCOO—, —NRCONR—, and —NRC(NR)NR—;
E′ is selected from a bond, an optionally substituted C₁-C₆ alkyl, optionally substituted C₁-C₆ alkenyl, and optionally substituted cycloalkyl; and
F′ is selected from H, an optionally substituted cycloalkyl, an optionally substituted non-aromatic heterocycle, an optionally substituted aryl, and an optionally substituted heteroaryl.

When R⁵ is an optionally substituted aryl, the moiety may be, e.g., a phenyl that is unsubstituted or substituted. In some embodiments, the phenyl may be substituted with one or more of alkyl, —OR, —CN, —CO₂R, halogen, —SR, —SOR, —SO₂R, —SF₅ and —NR₂.

When R⁵ is an optionally substituted heterocycle, the moiety may be, e.g., morpholine or pyridine, optionally substituted with one or more alkyl, —OR, —CN, —CO₂R, halogen, —SR, —SOR, —SO₂R, —SF₅ or —NR₂.

In some embodiments, R⁵ is C₁-C₆ alkyl optional substituted with alkoxy, hydroxy, amino, heterocyclyl, cycloalkyl, aryl, heteroaryl, or a combination of two or more thereof. In some embodiments, R⁵ is H.

In some embodiments of a compound of Formula (I′), when R⁵ is:

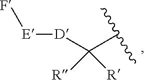

D′, E′ and F′ may be defined as follows:
D′ may be selected from the group consisting of —O—, —NR—, —OCONR—, —OCO—, —NRSO₂—, —NRCO—, —NRSO₂NR—, —NRCOO—, —NRCONR—, and —NRC(NR)NR—; E′ may be selected from the group consisting of a bond, an optionally substituted C₁-C₆ alkyl, optionally substituted C₁-C₆ alkenyl, and optionally substituted cycloalkyl; and F′ may be selected from the group consisting of H, halogen, an optionally substituted cycloalkyl, an optionally substituted non-aromatic heterocycle, an optionally substituted aryl, and an optionally substituted heteroaryl;
D′ is selected from the group consisting of —O—, —NR—, —OCONR—, —OCO—, —NRSO₂—, —NRCO—, —NRSO₂NR—, —NRCOO—, —NRCONR—, or —NRC(NR)NR—, where R is defined previously.

In some embodiments, D′ may be selected from the group consisting of O, NH, OCONH, OCO, NHSO₂, NHCO, NHSO₂NH, NHCOO, and NHCONH; and E′ may be a bond or an optionally substituted C₁-C₆ alkyl or cycloalkyl.

In some embodiments, E′ is an optionally substituted C₁-C₆ alkyl. In some embodiments, E′ is an optionally substituted C₁-C₆ alkyl and F′ is H. In some embodiments, E′ is C₁-C₆ alkyl substituted with a C₃-C₇ spirocycle. In some embodiments, E′ is an optionally substituted C₃-C₆ cycloalkyl, (e.g., optionally substituted with alkyl, halogen, or —OR). In some embodiments the cycloalkyl is specifically selected from, e.g., one of the following: cyclopropyl, cyclobutyl, cyclopentyl, and cyclohexyl, each of which may be optionally substituted.

F′ may be selected from the group consisting of H, an optionally substituted cycloalkyl, an optionally substituted non-aromatic heterocycle, an optionally substituted aryl, and an optionally substituted heteroaryl. In some embodiments, F′ is an optionally substituted cycloalkyl, an optionally substituted non-aromatic heterocycle, an optionally substituted aryl, or an optionally substituted heteroaryl, each of which is described below in further detail. In some embodiments, F′ is substituted with one or more alkyl, perfluoroalkyl (e.g., CF₃), —OR, —CN, —CO₂R, halogen, —SR, —SOR, —SO₂R, —SF₅ or —NR₂. In other embodiments, F′ is not substituted.

In some embodiments, F′ is an optionally substituted cycloalkyl. For example, F′ may be a C₃-C₆ cycloalkyl, optionally substituted with alkyl, halogen, or —OR. In some embodiments, the cycloalkyl is specifically selected from, e.g., one of the following: cyclopropyl, cyclobutyl, cyclopentyl, and cyclohexyl, each of which may be optionally substituted. In some embodiments, the cycloalkyl is specifically selected from, e.g., one of the following: cyclopropyl, cyclobutyl, cyclopentyl, cyclohexyl, and propellane, each of which may be optionally substituted. In some embodiments, E′ is a bond and F′ is an optionally substituted cycloalkyl. In some embodiments, E′ is optionally substituted C₁-C₆ alkyl and F′ is an optionally substituted cycloalkyl.

In some embodiments, F′ is an optionally substituted heterocycle. For example, F′ may be a 4-6 membered non-aromatic heterocycle, optionally substituted with alkyl, halogen, or OR. In some embodiments, the heterocycle contains one or more heteroatom selected from: O, S, SO, SO₂, B, N, and NR. Particular embodiments include, e.g., morpholine, azetidine, alkyl-piperidine, alkyl-piperazine, alkyl-diazepane, thiomorpholine 1,1-dioxide, isoindoline-1,3-dione, tetrahydropyran, and pyrrolidone. In some embodiments, E′ is a bond and F′ is an optionally substituted non-aromatic heterocycle. In some embodiments, E′ is optionally substituted C₁-C₆ alkyl and F′ is an optionally substituted non-aromatic heterocycle.

In some embodiments, F′ is an optionally substituted aryl. For example, F′ may be a C₆-C₁₀ aryl, e.g., a phenyl, that is optionally substituted, e.g., with one or more of alkyl, perfluoroalkyl (e.g., CF₃), —OR, —CN, —CO₂R, halogen, —SR, —SOR, —SO₂R, —SF₅ or —NR₂. In some embodiments, E′ is a bond and F′ is an optionally substituted aryl. In some embodiments, E′ is optionally substituted C₁-C₆ alkyl and F′ is an optionally substituted aryl.

In some embodiments, F′ is an optionally substituted heteroaryl. For example, F′ may be a 5-6 membered heteroaryl, optionally substituted with alkyl, halogen, or OR. In some embodiments, the heteroaryl contains one or more heteroatom selected from O, S, SO, N, and NR. Particular embodiments include, e.g., alkyl-triazole, tetrazole, imidazole, and isoxazole. In some embodiments, E′ is a bond and F′ is an optionally substituted heteroaryl. In some embodiments, E′ is optionally substituted C₁-C₆ alkyl and F′ is an optionally substituted heteroaryl.

In some embodiments of a compound of Formula (I′), when R⁵ is:

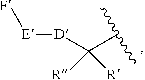

R′ and R″ may each independently selected from the group consisting of H, optionally substituted alkyl, —OR, halogen (e.g., F, Cl, Br, I), —N(R)₂, optionally substituted cycloalkyl, and optionally substituted heterocycle. In some embodiments, R′ and R″ are each H. In other embodiments, at least one of R′ and R″ is optionally substituted alkyl and any remaining R′ or R″ is H. In some embodiments, the optionally substituted alkyl is a C₁-C₆ alkyl or C₁-C₆ perfluoroalkyl. In some embodiments, the optionally substituted alkyl is methyl. In some embodiments, R′ and R″ are each independently optionally substituted C₁-C₆ alkyl.

Alternatively, R′ and R″ together may form a 3- to 6-membered cycloalkyl or heterocycle that is optionally substituted. In some embodiments, the 3- to 6-membered cycloalkyl or heterocycle is substituted by one or more R⁷, as defined previously. In additional embodiments, R′ and R″ form a cyclopropyl, cyclobutyl, cyclopentyl, or cyclohexyl. In other embodiments, R′ and R″ form an oxetane. In some embodiments, R′ and R″ form an optionally substituted azetidine, oxetane, pyrrolidone or piperidine. In some embodiments, the nitrogen of the azetidine, pyrrolidone or piperidine may be substituted with R, SO₂R, COR, SO₂NR₂, CONR₂ and COOR. In some embodiments, when R′ and R″ form a ring, e.g., an optionally substituted cyclopropyl, cyclobutyl, cyclopentyl, or cyclohexyl, azetidine, oxetane, pyrrolidone or piperidine, then D′-E′-F′ together form an —OH (i.e., D′ is —O—, E′ is a bond and F′ is H).

In some embodiments of a compound of Formula (I′), R¹ is an optionally substituted phenyl or an optionally substituted 5- or 6-membered heteroaryl. For example, in some embodiments, R¹ is optionally substituted phenyl or optionally substituted pyridine. In some embodiments, R¹ is an unsubstituted phenyl or an unsubstituted pyridine. In some embodiments, R¹ is a pyridine. In some embodiments the pyridine is attached at the 2 position, the 3 position or the 4 position. In some embodiments the pyridine is attached at the 3 position.

In some embodiments of a compound of Formula (I′), R² and R³ are independently an optionally substituted C₁₋₉ cyclic, optionally substituted C₃₋₉ heterocyclic, or halogen, or R² and R³ together form an optionally substituted C₃₋₉ cyclic or optionally substituted 3- to 9-membered heterocyclic ring. In some embodiments, R² is a C₁-C₆ alkyl, a C₃-C₁₀ cycloalkyl (including cycloalkenyl), or a heterocycle, each of which maybe optionally substituted.

In some embodiments, R² and R³ together form an optionally substituted C₃₋₉ cyclic or optionally substituted 3- to 9-membered heterocyclic ring, for example, a C₃-C₁₀ cycloalkyl (including cycloalkenyl), or a heterocycle, each of which maybe optionally substituted.

Exemplary individual embodiments when R² and R³ together form an optionally substituted C₃₋₉ cyclic or optionally substituted 3- to 9-membered heterocyclic ring, include cyclopropyl, cyclobutyl, cyclopentyl, cyclohexyl, cyclopentene, and cyclohexene, each of which may be optionally substituted, for example, by one or more deuterium or fluorine moiety. Other embodiments include bicyclic structures, such as:

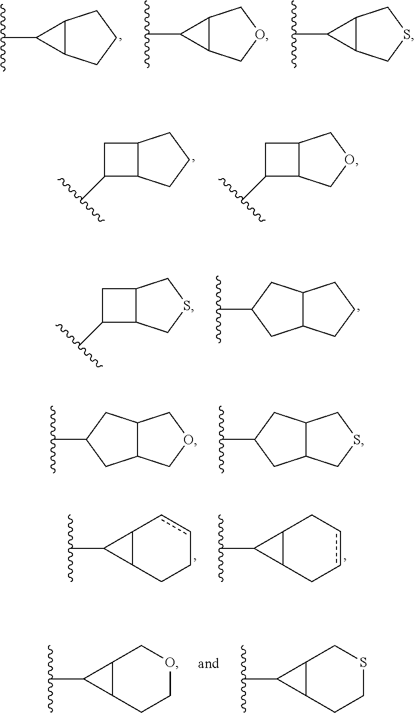

where one or more of the dashed bonds may optionally be a double bond and [symbol] indicates attachment to Z.

In some embodiments, one or more hydrogens in R² or R³, or in both R² and R³ are replaced with deuterium. In some embodiments, R² and R³ form a perdeuterated cycloalkyl ring.

In some embodiments, R² and R³ form a perdeuterated cyclopentane

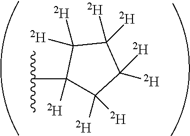

and [symbol] indicates attachment to Z. In some embodiments,

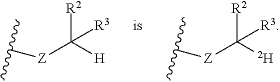

In some embodiments, compounds of the present disclosure include those represented by the following Formula (II):

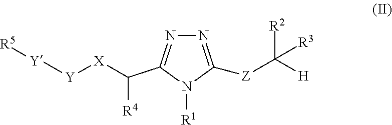

or a pharmaceutically acceptable salt, solvate or prodrug thereof, wherein:

### X is O or SO₀₋₂ or NH;

Y is an optionally substituted alkyl, optionally substituted alkenyl, alkynyl, optionally substituted aryl, optionally substituted heteroaryl, optionally substituted cycloalkyl, or optionally substituted non-aromatic heterocycle;
Y′ is an alkynyl, optionally substituted aryl, optionally substituted heteroaryl, optionally substituted cycloalkyl, or optionally substituted non-aromatic heterocycle;
R² and R³ are independently optionally substituted C₁₋₆ alkyl, optionally substituted C₁₋₉ cyclic, optionally substituted C₃₋₉ heterocyclic, or halogen, or R² and R³ together form an optionally substituted C₃₋₉ cyclic or optionally substituted 3- to 9-membered heterocyclic ring; and
the remaining variables have the same values as for Formula (I′).

In some embodiments of a compound of Formula (II), Y and Y′ are not both phenylene. In some embodiments, Y′ is selected from the group consisting of an alkyne, an optionally substituted heteroaryl, an optionally substituted C₃-C₁₀ cycloalkyl, and an optionally substituted non-aromatic heterocycle (containing one or more O, S, SO, SO₂, N, or NR). In some embodiments, Y′ is selected from the group consisting of:

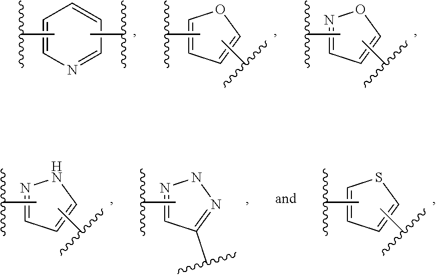

each of which may be optionally substituted. In some embodiments, Y′ is cyclobutene, propellane (such as, but not limited to, [1.1.1.0¹³] propellane), cubane or

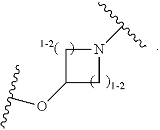

In some embodiments, Y is selected from the group consisting of an optionally substituted alkyl, an optionally substituted alkenyl, an optionally substituted aryl, an optionally substituted heteroaryl, an optionally substituted C₃-C₁₀ cycloalkyl, and an optionally substituted non-aromatic heterocycle (containing one or more O, S, SO, SO₂, B, N, or NR). In some embodiments, Y is selected from:

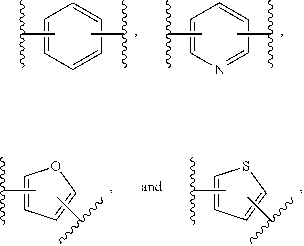

each of which may be optionally substituted. In some embodiments, Y is

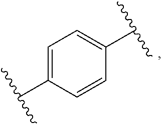 [SMILES: *c1ccc(*)cc1]

which may be optionally substituted, for example the phenylene may be substituted alkyl or cycloalkyl, halogen, —NR₂, —SF₅ and —OR. In some embodiments, the phenylene is substituted by methyl or perfluoromethyl. In some embodiments, the substitution is at a position ortho to the alkyne. Other examples within these embodiments, include phenylene moieties substituted by one or more fluoro moiety. For example, the phenylene moieties may be

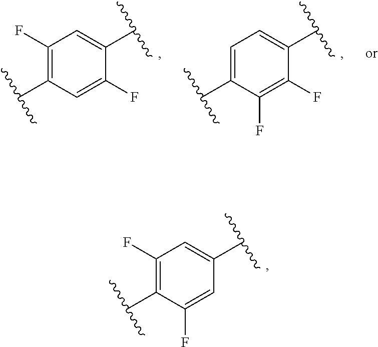 [SMILES: *c1cc(F)c(*)cc1F ; *c1ccc(*)c(F)c1F ; *c1cc(F)c(*)c(F)c1]

or further substituted variants thereof, e.g.,

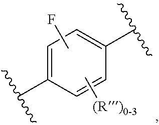

where R′″ is a halogen (e.g., F, Cl, or Br), nitrile, a C₁-C₆ alkyl, or O—C₁-C₆ alkyl. In some embodiments, the phenylene is di-substituted, for example,

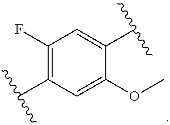 [SMILES: *c1cc(OC)c(*)cc1F]

In some embodiments, Y is phenylene optionally substituted with C₁-C₆ alkyl, C₁-C₆ haloalkyl, halogen, C₁-C₆ alkoxy, nitrile, or a combination of two or more thereof.

In some embodiments, Y is cyclobutene, propellane (such as, but not limited to, [1.1.1.0¹³] propellane), cubane or

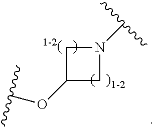

In Formula (II), Z is O, SO₀₋₂, NR, C(R⁷)₂, alkenyl or alkynyl. In some embodiments, Z is selected from the group consisting of O, S, and C(R⁷)₂, where R⁷ is independently H, halogen, optionally substituted alkyl, optionally substituted cycloalkyl, or —N(R)₂. In some embodiments, R or R⁷ is H. In some embodiments, Z is S.

In Formula (II), R⁵ is H, nitrile, an optionally substituted aryl, an optionally substituted heterocycle, optionally substituted C₁-C₆ alkyl, optionally substituted C₃-C₉ cycloalkyl, or

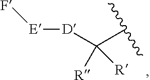

where R′ and R″ are each independently selected from the group consisting of H, optionally substituted alkyl, —OR, halogen (e.g., F, Cl, Br, or I), —N(R)₂, optionally substituted cycloalkyl, and optionally substituted heterocycle;
or R′ and R″ may together form an optionally substituted 3- to 6-membered cycloalkyl or optionally substituted 3- to 6-membered heterocycle;
D′ is selected from the group consisting of —O—, —NR—, —OCONR—, —OCO—, —NRSO₂—, —NRCO—, —NRSO₂NR—, —NRCOO—, —NRCONR—, and —NRC(NR)NR—;
E′ is selected from the group consisting of a bond, an optionally substituted C₁-C₆ alkyl, optionally substituted C₁-C₆ alkenyl, and optionally substituted cycloalkyl; and
F′ is selected from the group consisting of H, an optionally substituted cycloalkyl, an optionally substituted non-aromatic heterocycle, an optionally substituted aryl, and an optionally substituted heteroaryl.

When R⁵ is an optionally substituted aryl, the moiety may be, e.g., a phenyl that is unsubstituted or substituted. In some embodiments, the phenyl may substituted with one or more of alkyl, —OR, —CN, —CO₂R, halogen, —SR, —SOR, —SO₂R, —SF₅ or —NR₂.

When R⁵ is an optionally substituted heterocycle, the moiety may be, e.g., morpholine or pyridine, optionally substituted with one or more alkyl, —OR, —CN, —CO₂R, halogen, —SR, —SOR, —SO₂R, —SF₅ or —NR₂.

In some embodiments, R⁵ is C₁-C₆ alkyl optional substituted with alkoxy, hydroxy, amino, heterocyclyl, cycloalkyl, aryl, heteroaryl, or a combination of two or more thereof. In some embodiments, R⁵ is H.

In some embodiments of a compound of Formula (II), when R⁵ is:

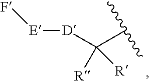

D′, E′ and F′ may be defined as follows:

D′ may be selected from the group consisting of —O—, —NR—, —OCONR—, —OCO—, —NRSO₂—, —NRCO—, —NRSO₂NR—, —NRCOO—, —NRCONR—, and —NRC(NR)N—;

E′ may be selected from the group consisting of a bond, an optionally substituted C₁-C₆ alkyl, optionally substituted C₁-C₆ alkenyl, and optionally substituted cycloalkyl; and F′ may be selected from the group consisting of H, halogen, an optionally substituted cycloalkyl, an optionally substituted non-aromatic heterocycle, an optionally substituted aryl, and an optionally substituted heteroaryl.

In some embodiments, D′ is selected from the group consisting of —O—, —NR—, —OCONR—, —OCO—, —NRSO₂—, —NRCO—, —NRSO₂NR—, —NRCOO—, —NRCONR—, and —NRC(NR)NR—, where R is defined previously. In some embodiments, D′ may be selected from the group consisting of O, NH, OCONH, OCO, NHSO₂, NHCO, NHSO₂NH, NHCOO, and NHCONH.

In some embodiments, E′ may be a bond or an optionally substituted C₁-C₆ alkyl or cycloalkyl. In some embodiments, E′ is an optionally substituted C₁-C₆ alkyl. In some embodiments, E′ is an optionally substituted C₁-C₆ alkyl and F′ is H. In some embodiments, E′ is C₁-C₆ alkyl substituted with a C₃-C₇ spirocycle. In some embodiments, E′ is an optionally substituted C₃-C₆ cycloalkyl, (e.g., optionally substituted with alkyl, halogen, or —OR). In some embodiments, the cycloalkyl is specifically selected from, e.g., one of the following: cyclopropyl, cyclobutyl, cyclopentyl, and cyclohexyl, each of which may be optionally substituted.

In some embodiments, F′ may be selected from the group consisting of H, an optionally substituted cycloalkyl, an optionally substituted non-aromatic heterocycle, an optionally substituted aryl, and an optionally substituted heteroaryl. In some embodiments, F′ is an optionally substituted cycloalkyl, an optionally substituted non-aromatic heterocycle, an optionally substituted aryl, or an optionally substituted heteroaryl, each of which is described below in further detail. In some embodiments, F′ is substituted with one or more alkyl, perfluoroalkyl (e.g., CF₃), —OR, —CN, —CO₂R, halogen, —SR, —SOR, —SO₂R, —SF₅ or —NR₂. In other embodiments, F′ is not substituted.

In some embodiments, F′ is an optionally substituted cycloalkyl. For example, F′ may be a C₃-C₆ cycloalkyl, optionally substituted with alkyl, halogen, or —OR. In some embodiments, the cycloalkyl is specifically selected from, e.g., one of the following: cyclopropyl, cyclobutyl, cyclopentyl, and cyclohexyl, each of which may be optionally substituted. In some embodiments, the cycloalkyl is specifically selected from, e.g., one of the following: cyclopropyl, cyclobutyl, cyclopentyl, cyclohexyl, and propellane, each of which may be optionally substituted. In some embodiments, E′ is a bond and F′ is an optionally substituted cycloalkyl. In some embodiments, E′ is optionally substituted C₁-C₆ alkyl and F′ is an optionally substituted cycloalkyl.

In some embodiments, F′ is an optionally substituted heterocycle. For example, F′ may be a 4-6 membered non-aromatic heterocycle, optionally substituted with alkyl, halogen, or OR. In some embodiments, the heterocycle contains one or more heteroatom selected from: O, S, SO, SO₂, B, N, and NR. Particular embodiments include, e.g., morpholine, azetidine, alkyl-piperidine, alkyl-piperazine, alkyl-diazepane, thiomorpholine 1,1-dioxide, isoindoline-1,3-dione, tetrahydropyran, and pyrrolidone. In some embodiments, E′ is a bond and F′ is an optionally substituted non-aromatic heterocycle. In some embodiments, E′ is optionally substituted C₁-C₆ alkyl and F′ is an optionally substituted non-aromatic heterocycle.

In some embodiments, F′ is an optionally substituted aryl. For example, F′ may be a C₆-C₁₀ aryl, e.g., a phenyl, that is optionally substituted, e.g., with one or more of alkyl, perfluoroalkyl (e.g., CF₃), —OR, —CN, —CO₂R, halogen, —SR, —SOR, —SO₂R, —SF₅ or —NR₂. In some embodiments, E′ is a bond and F′ is an optionally substituted aryl. In some embodiments, E′ is optionally substituted C₁-C₆ alkyl and F′ is an optionally substituted aryl.

In some embodiments, F′ is an optionally substituted heteroaryl. For example, F′ may be a 5-6 membered heteroaryl, optionally substituted with alkyl, halogen, or OR. In some embodiments, the heteroaryl contains one or more heteroatom selected from O, S, SO, N, and NR. Particular embodiments include, e.g., alkyl-triazole, tetrazole, imidazole, and isoxazole. In some embodiments, E′ is a bond and F′ is an optionally substituted heteroaryl. In some embodiments, E′ is optionally substituted C₁-C₆ alkyl and F′ is an optionally substituted heteroaryl.

In some embodiments of a compound of Formula (II), when R⁵ is:

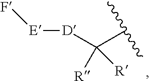

R′ and R″ may each independently selected from the group consisting of H, optionally substituted alkyl, —OR, halogen (e.g., F, Cl, Br, I), —N(R)₂, optionally substituted cycloalkyl, and optionally substituted heterocycle. In some embodiments, R′ and R″ are each H. In other embodiments, at least one of R′ and R″ is optionally substituted alkyl and any remaining R′ or R″ is H. In some embodiments, the optionally substituted alkyl is a C₁-C₆ alkyl or C₁-C₆ perfluoroalkyl. In some embodiments, the optionally substituted alkyl is methyl. In some embodiments, R′ and R″ are each independently optionally substituted C₁-C₆ alkyl.

Alternatively, R′ and R″ together may form a 3- to 6-membered cycloalkyl or heterocycle that is optionally substituted. In some embodiments, the 3- to 6-membered cycloalkyl or heterocycle is substituted by one or more R⁷, as defined previously. In additional embodiments, R′ and R″ form a cyclopropyl, cyclobutyl, cyclopentyl, or cyclohexyl. In other embodiments, R′ and R″ form an oxetane. In some embodiments, R′ and R″ form an optionally substituted azetidine, oxetane, pyrrolidone or piperidine. In some embodiments, the nitrogen of the azetidine, pyrrolidone or piperidine may be substituted with R, SO₂R, COR, SO₂NR₂, CONR₂ or COOR. In some embodiments, when R′ and R″ form a ring, e.g., an optionally substituted cyclopropyl, cyclobutyl, cyclopentyl, or cyclohexyl, azetidine, oxetane, pyrrolidone or piperidine, then D′-E′-F′ together form an —OH (i.e., D′ is —O—, E′ is a bond and F′ is H).

In some embodiments of a compound of Formula (II), R¹ is an optionally substituted phenyl or an optionally substituted 5- or 6-membered heteroaryl. For example, in some embodiments, R¹ is optionally substituted phenyl or optionally substituted pyridine. In some embodiments, R¹ is an unsubstituted phenyl or an unsubstituted pyridine. In some embodiments, R¹ is a pyridine. In some embodiments the pyridine is attached at the 2 position, the 3 position or the 4 position. In some embodiments the pyridine is attached at the 3 position.

In some embodiments of a compound of Formula (II), R² and R³ are independently an optionally substituted C₁₋₉ cyclic, optionally substituted C₃₋₉ heterocyclic, or halogen, or R² and R³ together form an optionally substituted C₃₋₉ cyclic or optionally substituted 3- to 9-membered heterocyclic ring. In some embodiments, R² is a C₁-C₆ alkyl, a C₃-C₁₀ cycloalkyl (including cycloalkenyl), or a heterocycle, each of which maybe optionally substituted. In some embodiments, R² is optionally substituted C₁₋₆ alkyl. In some embodiments, R² is C₁₋₆ alkyl substituted with a C₃₋₉ heterocyclic group, a C₁₋₉ cyclic group, or one or more alkenes.

In some embodiments, R² and R³ together form an optionally substituted C₃₋₉ cyclic or optionally substituted 3- to 9-membered heterocyclic ring, for example, a C₃-C₁₀ cycloalkyl (including cycloalkenyl), or a heterocycle, each of which maybe optionally substituted.

Exemplary individual embodiments when R² and R³ together form an optionally substituted C₃₋₉ cyclic or optionally substituted 3- to 9-membered heterocyclic ring, include cyclopropyl, cyclobutyl, cyclopentyl, cyclohexyl, cyclopentene, and cyclohexene, each of which may be optionally substituted, for example, by one or more deuterium or fluorine moiety. Other embodiments include bicyclic structures, such as:

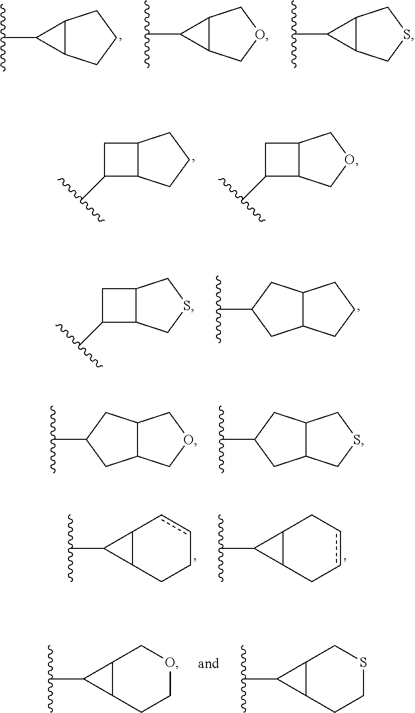

where one or more of the dashed bonds may optionally be a double bond and [symbol] indicates attachment to Z.

In some embodiments, one or more hydrogens in R² or R³, or in both R² and R³ are replaced with deuterium. In some embodiments, R² and R³ form a perdeuterated cycloalkyl ring. In some embodiments, R² and R³ form a perdeuterated cyclopentane

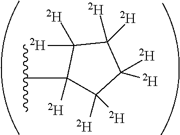

and [symbol] indicates attachment to Z. In some embodiments,

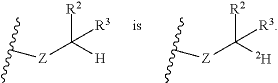

In some embodiments, the compounds of the present disclosure are selected from the compounds of Table I or a pharmaceutically acceptable salt or prodrug thereof.

TABLE I

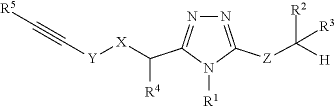

| | | | | | | | |
|---|---|---|---|---|---|---|---|
| R5 | Y | X | R⁴ | R1 | Z | R² | R³ |

| | | | | | | |
|---|---|---|---|---|---|---|
| H | alkyl | O | H | optionally | S | a C₃₋₉ cyclic or C₃₋₉ |
| | alkenyl | | | substituted 5- or | | heterocyclic ring |
| | | | | 6-membered | | |
| | | | | heteroaryl | | |
| nitrile | alkyl or | O | H | optionally | S | a C₃₋₉ cyclic or C₃₋₉ |
| | alkenyl | | | substituted 5- or | | heterocyclic ring |
| | | | | 6-membered | | |
| | | | | heteroaryl | | |
| optionally | alkyl or | O | H | optionally | S | a C₃₋₉ cyclic or C₃₋₉ |
| substituted | alkenyl | | | substituted 5- or | | heterocyclic ring |
| aryl | | | | 6-membered | | |
| | | | | heteroaryl | | |
| optionally | alkyl or | O | H | optionally | S | a C₃₋₉ cyclic or C₃₋₉ |
| substituted | alkenyl | | | substituted 5- or | | heterocyclic ring |
| heterocycle | | | | 6-membered | | |
| | | | | heteroaryl | | |
| optionally | alkyl or | O | H | optionally | S | a C₃₋₉ cyclic or C₃₋₉ |
| substituted | alkenyl | | | substituted 5- or | | heterocyclic ring |
| C₁₋₆ alkyl | | | | 6-membered | | |
| | | | | heteroaryl | | |
| optionally | alkyl or | O | H | optionally | S | a C₃₋₉ cyclic or C₃₋₉ |
| substituted | alkenyl | | | substituted 5- or | | heterocyclic ring |
| C₃₋₉ | | | | 6-membered | | |
| cycloalkyl | | | | heteroaryl | | |
| 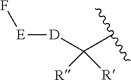 | alkyl or alkenyl | O | H | optionally substituted 5- or 6-membered heteroaryl | S | a C₃₋₉ cyclic or C₃₋₉ heterocyclic ring |
| H | optionally | O | H | optionally | S | an optionally substituted C₃₋₉ |
| | substituted | | | substituted 5- or | | cyclic or optionally substituted |
| | aryl | | | 6-membered | | C₃₋₉ heterocyclic ring |
| | | | | heteroaryl | | |
| nitrile | optionally | O | H | optionally | S | an optionally substituted C₃₋₉ |
| | substituted | | | substituted 5- or | | cyclic or optionally substituted |
| | aryl | | | 6-membered | | C₃₋₉ heterocyclic ring |
| | | | | heteroaryl | | |
| optionally | optionally | O | H | optionally | S | an optionally substituted C₃₋₉ |
| substituted | substituted | | | substituted 5- or | | cyclic or optionally substituted |
| aryl | aryl | | | 6-membered | | C₃₋₉ heterocyclic ring |
| | | | | heteroaryl | | |
| optionally | optionally | O | H | optionally | S | an optionally substituted C₃₋₉ |
| substituted | substituted | | | substituted 5- or | | cyclic or optionally substituted |
| heterocycle | aryl | | | 6-membered | | C₃₋₉ heterocyclic ring |
| | | | | heteroaryl | | |
| optionally | optionally | O | H | optionally | S | an optionally substituted C₃₋₉ |
| substituted | substituted | | | substituted 5- or | | cyclic or optionally substituted |
| C₁₋₆ alkyl | aryl | | | 6-membered | | C₃₋₉ heterocyclic ring |
| | | | | heteroaryl | | |
| optionally | optionally | O | H | optionally | S | an optionally substituted C₃₋₉ |
| substituted | substituted | | | substituted 5- or | | cyclic or optionally substituted |
| C₃₋₉ | aryl | | | 6-membered | | C₃₋₉ heterocyclic ring |
| cycloalkyl | | | | heteroaryl | | |
| 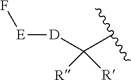 | optionally substituted aryl | O | H | optionally substituted 5- or 6-membered heteroaryl | S | an optionally substituted C₃₋₉ cyclic or optionally substituted C₃₋₉ heterocyclic ring |

In some embodiments, when R⁵ is

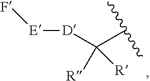

the moiety can be, for example, one of the following of Table II. In some embodiments, one or more of the moieties in Table II are substituted (e.g., E′ is an optionally substituted C₁-C₆ alkyl).

TABLE II

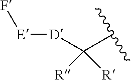

| F′ | E′ | D′ | R′ | R″ |
|---|---|---|---|---|
| H | C₁₋₆ alkyl | —O— | H | H |
| H | C₁₋₆ alkyl | —NR— | H | H |
| H | C₁₋₆ alkyl | —OCONR— | H | H |
| H | C₁₋₆ alkyl | —OCO— | H | H |
| H | C₁₋₆ alkyl | —NRSO₂— | H | H |
| H | C₁₋₆ alkyl | —NRCO— | H | H |
| H | C₁₋₆ alkyl | —NRSO₂NR— | H | H |
| H | C₁₋₆ alkyl | —NRCOO— | H | H |
| H | C₁₋₆ alkyl | —NRCONR— | H | H |
| H | C₁₋₆ alkyl | —NRC(NR)NR— | H | H |
| optionally substituted cycloalkyl | C₁₋₆ alkyl | —O— | H | H |
| optionally substituted cycloalkyl | C₁₋₆ alkyl | —NR— | H | H |
| optionally substituted cycloalkyl | C₁₋₆ alkyl | —OCONR— | H | H |
| optionally substituted cycloalkyl | C₁₋₆ alkyl | —OCO— | H | H |
| optionally substituted cycloalkyl | C₁₋₆ alkyl | —NRSO₂— | H | H |
| optionally substituted cycloalkyl | C₁₋₆ alkyl | —NRCO— | H | H |
| optionally substituted cycloalkyl | C₁₋₆ alkyl | —NRSO₂NR— | H | H |
| optionally substituted cycloalkyl | C₁₋₆ alkyl | —NRCOO— | H | H |
| optionally substituted cycloalkyl | C₁₋₆ alkyl | —NRCONR— | H | H |
| optionally substituted cycloalkyl | C₁₋₆ alkyl | —NRC(NR)NR— | H | H |
| optionally substituted heterocycle | C₁₋₆ alkyl | —O— | H | H |
| optionally substituted heterocycle | C₁₋₆ alkyl | —NR— | H | H |
| optionally substituted heterocycle | C₁₋₆ alkyl | —OCONR— | H | H |
| optionally substituted heterocycle | C₁₋₆ alkyl | —OCO— | H | H |
| optionally substituted heterocycle | C₁₋₆ alkyl | —NRSO₂— | H | H |
| optionally substituted heterocycle | C₁₋₆ alkyl | —NRCO— | H | H |
| optionally substituted heterocycle | C₁₋₆ alkyl | —NRSO₂NR— | H | H |
| optionally substituted heterocycle | C₁₋₆ alkyl | —NRCOO— | H | H |
| optionally substituted heterocycle | C₁₋₆ alkyl | —NRCONR— | H | H |
| optionally substituted heterocycle | C₁₋₆ alkyl | —NRC(NR)NR— | H | H |
| optionally substituted aryl | C₁₋₆ alkyl | —O— | H | H |
| optionally substituted aryl | C₁₋₆ alkyl | —NR— | H | H |
| optionally substituted aryl | C₁₋₆ alkyl | —OCONR— | H | H |
| optionally substituted aryl | C₁₋₆ alkyl | —OCO— | H | H |
| optionally substituted aryl | C₁₋₆ alkyl | —NRSO₂— | H | H |
| optionally substituted aryl | C₁₋₆ alkyl | —NRCO— | H | H |
| optionally substituted aryl | C₁₋₆ alkyl | —NRSO₂NR— | H | H |
| optionally substituted aryl | C₁₋₆ alkyl | —NRCOO— | H | H |
| optionally substituted aryl | C₁₋₆ alkyl | —NRCONR— | H | H |
| optionally substituted aryl | C₁₋₆ alkyl | —NRC(NR)NR— | H | H |
| optionally substituted heteroaryl | C₁₋₆ alkyl | —O— | H | H |
| optionally substituted heteroaryl | C₁₋₆ alkyl | —NR— | H | H |
| optionally substituted heteroaryl | C₁₋₆ alkyl | —OCONR— | H | H |
| optionally substituted heteroaryl | C₁₋₆ alkyl | —OCO— | H | H |
| optionally substituted heteroaryl | C₁₋₆ alkyl | —NRSO₂— | H | H |
| optionally substituted heteroaryl | C₁₋₆ alkyl | —NRCO— | H | H |
| optionally substituted heteroaryl | C₁₋₆ alkyl | —NRSO₂NR— | H | H |
| optionally substituted heteroaryl | C₁₋₆ alkyl | —NRCOO— | H | H |
| optionally substituted heteroaryl | C₁₋₆ alkyl | —NRCONR— | H | H |
| optionally substituted heteroaryl | C₁₋₆ alkyl | —NRC(NR)NR— | H | H |
| optionally substituted cycloalkyl | bond | —O— | H | H |
| optionally substituted cycloalkyl | bond | —NR— | H | H |
| optionally substituted cycloalkyl | bond | —OCONR— | H | H |
| optionally substituted cycloalkyl | bond | —OCO— | H | H |
| optionally substituted cycloalkyl | bond | —NRSO₂— | H | H |
| optionally substituted cycloalkyl | bond | —NRCO— | H | H |
| optionally substituted cycloalkyl | bond | —NRSO₂NR— | H | H |
| optionally substituted cycloalkyl | bond | —NRCOO— | H | H |
| optionally substituted cycloalkyl | bond | —NRCONR— | H | H |
| optionally substituted cycloalkyl | bond | —NRC(NR)NR— | H | H |
| optionally substituted heterocycle | bond | —NR— | H | H |
| optionally substituted heterocycle | bond | —OCONR— | H | H |
| optionally substituted heterocycle | bond | —OCO— | H | H |
| optionally substituted heterocycle | bond | —NRSO₂— | H | H |
| optionally substituted heterocycle | bond | —NRCO— | H | H |
| optionally substituted heterocycle | bond | —NRSO₂NR— | H | H |
| optionally substituted heterocycle | bond | —NRCOO— | H | H |
| optionally substituted heterocycle | bond | —NRCONR— | H | H |
| optionally substituted heterocycle | bond | —NRC(NR)NR— | H | H |
| optionally substituted aryl | bond | —NR— | H | H |
| optionally substituted aryl | bond | —OCONR— | H | H |
| optionally substituted aryl | bond | —OCO— | H | H |

In some embodiments, the compounds of the present disclosure are selected from the compounds of Table III (shown below) or a pharmaceutically acceptable salt or prodrug thereof. It should be noted that the moieties of the compounds of Table III fall within the scope of compounds of Formulae (I), (I′), or (II). The present disclosure includes embodiments where one or more of the variable moieties of Formulae (I), (I′), or (II) are represented by the equivalent moiety of one or more of the compounds of Table III without requiring the other specific moieties of the same compound of Table III.

Additional species which can be used in the methods and compositions described herein are shown below in Table III and can be found in WO2017/197080, as well as prepared according to the methods described thereby.

TABLE III

| No | Compound |
|---|---|
| 1 | 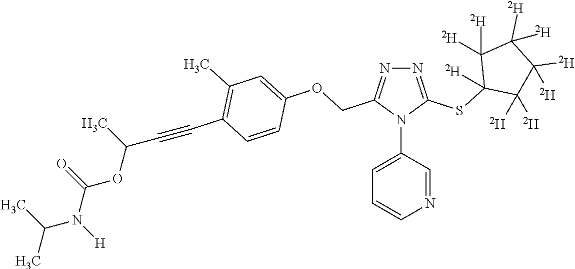 [SMILES: [2H]C1([2H])C([2H])([2H])C([2H])([2H])C([2H])(Sc2nnc(COc3ccc(C#CC(C)OC(=O)NC(C)C)c(C)c3)n2-c2cccnc2)C1([2H])[2H]] |
| 2 | 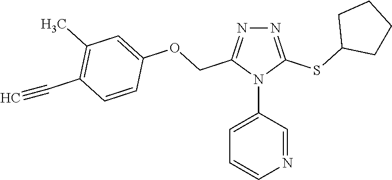 [SMILES: C#Cc1ccc(OCc2nnc(SC3CCCC3)n2-c2cccnc2)cc1C] |
| 3 | 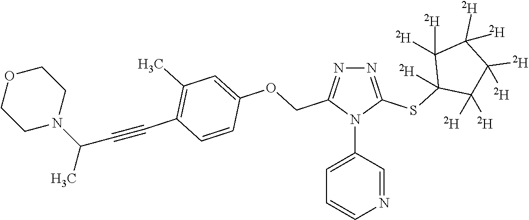 [SMILES: [2H]C1([2H])C([2H])([2H])C([2H])([2H])C([2H])(Sc2nnc(COc3ccc(C#CC(C)N4CCOCC4)c(C)c3)n2-c2cccnc2)C1([2H])[2H]] |
| 4 | 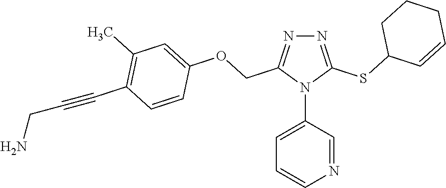 [SMILES: Cc1cc(OCc2nnc(SC3C=CCCC3)n2-c2cccnc2)ccc1C#CCN] |
| 5 | 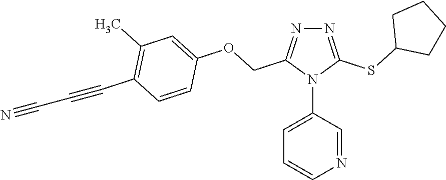 [SMILES: Cc1cc(OCc2nnc(SC3CCCC3)n2-c2cccnc2)ccc1C#CC#N] |
| 6 | 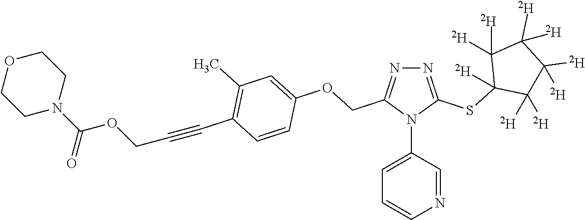 [SMILES: [2H]C1([2H])C([2H])([2H])C([2H])([2H])C([2H])(Sc2nnc(COc3ccc(C#CCOC(=O)N4CCOCC4)c(C)c3)n2-c2cccnc2)C1([2H])[2H]] |
| 7 | 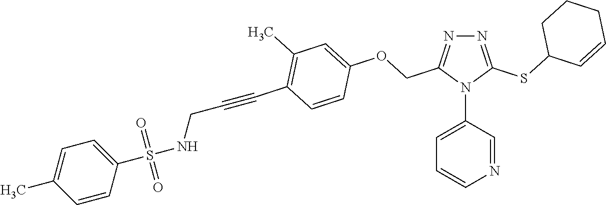 [SMILES: Cc1ccc(S(=O)(=O)NCC#Cc2ccc(OCc3nnc(SC4C=CCCC4)n3-c3cccnc3)cc2C)cc1] |
| 8 | 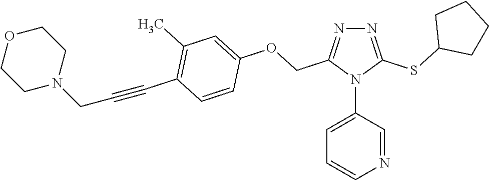 [SMILES: Cc1cc(OCc2nnc(SC3CCCC3)n2-c2cccnc2)ccc1C#CCN1CCOCC1] |
| 9 | 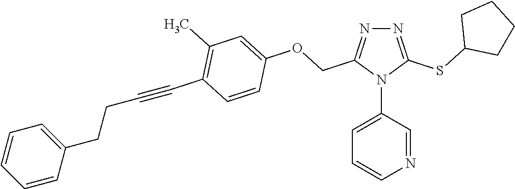 [SMILES: Cc1cc(OCc2nnc(SC3CCCC3)n2-c2cccnc2)ccc1C#CCCc1ccccc1] |
| 10 | 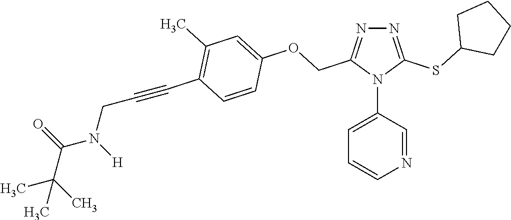 [SMILES: Cc1cc(OCc2nnc(SC3CCCC3)n2-c2cccnc2)ccc1C#CCNC(=O)C(C)(C)C] |
| 11 | 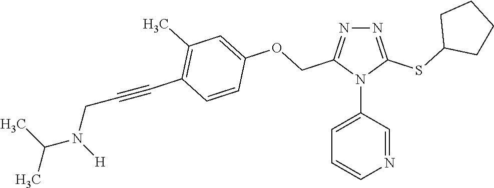 [SMILES: Cc1cc(OCc2nnc(SC3CCCC3)n2-c2cccnc2)ccc1C#CCNC(C)C] |
| 12 | 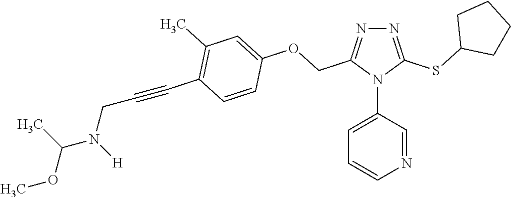 [SMILES: COC(C)NCC#Cc1ccc(OCc2nnc(SC3CCCC3)n2-c2cccnc2)cc1C] |
| 13 | 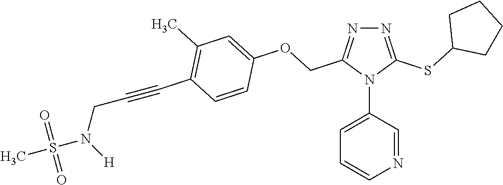 [SMILES: Cc1cc(OCc2nnc(SC3CCCC3)n2-c2cccnc2)ccc1C#CCNS(C)(=O)=O] |
| 14 | 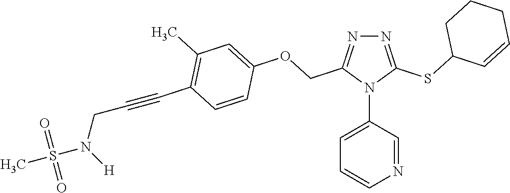 [SMILES: Cc1cc(OCc2nnc(SC3C=CCCC3)n2-c2cccnc2)ccc1C#CCNS(C)(=O)=O] |
| 15 | 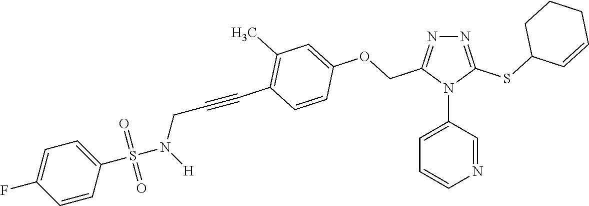 [SMILES: Cc1cc(OCc2nnc(SC3C=CCCC3)n2-c2cccnc2)ccc1C#CCNS(=O)(=O)c1ccc(F)cc1] |
| 16 | 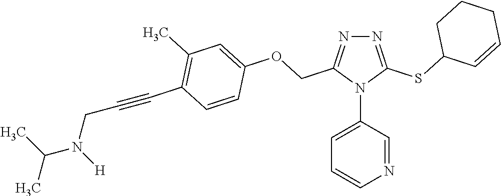 [SMILES: Cc1cc(OCc2nnc(SC3C=CCCC3)n2-c2cccnc2)ccc1C#CCNC(C)C] |
| 17 | 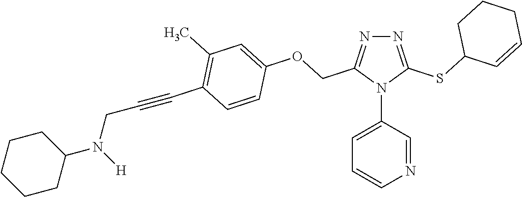 [SMILES: Cc1cc(OCc2nnc(SC3C=CCCC3)n2-c2cccnc2)ccc1C#CCNC1CCCCC1] |
| 18 | 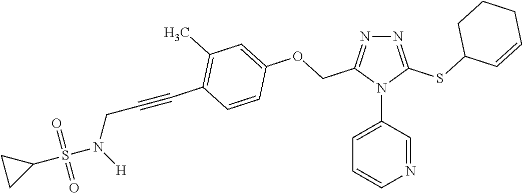 [SMILES: Cc1cc(OCc2nnc(SC3C=CCCC3)n2-c2cccnc2)ccc1C#CCNS(=O)(=O)C1CC1] |
| 19 | 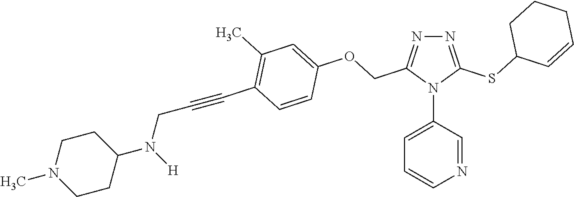 [SMILES: Cc1cc(OCc2nnc(SC3C=CCCC3)n2-c2cccnc2)ccc1C#CCNC1CCN(C)CC1] |
| 20 | 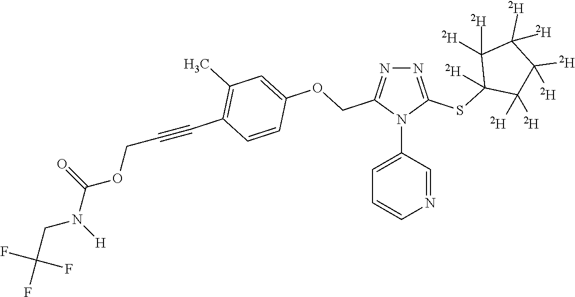 [SMILES: [2H]C1([2H])C([2H])([2H])C([2H])([2H])C([2H])(Sc2nnc(COc3ccc(C#CCOC(=O)NCC(F)(F)F)c(C)c3)n2-c2cccnc2)C1([2H])[2H]] |
| 21 | 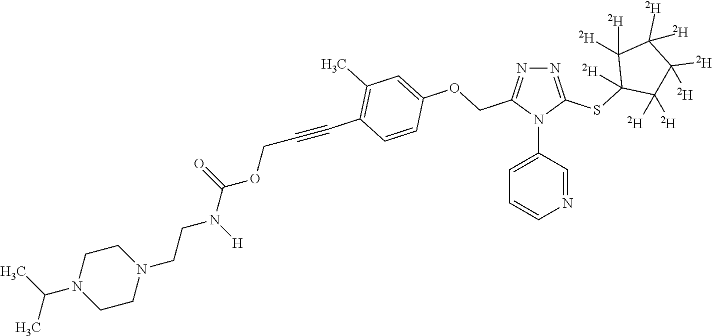 [SMILES: [2H]C1([2H])C([2H])([2H])C([2H])([2H])C([2H])(Sc2nnc(COc3ccc(C#CCOC(=O)NCCN4CCN(C(C)C)CC4)c(C)c3)n2-c2cccnc2)C1([2H])[2H]] |
| 22 | 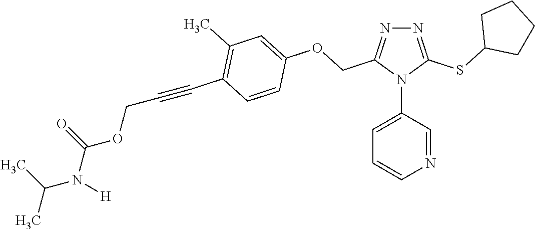 [SMILES: Cc1cc(OCc2nnc(SC3CCCC3)n2-c2cccnc2)ccc1C#CCOC(=O)NC(C)C] |
| 23 | 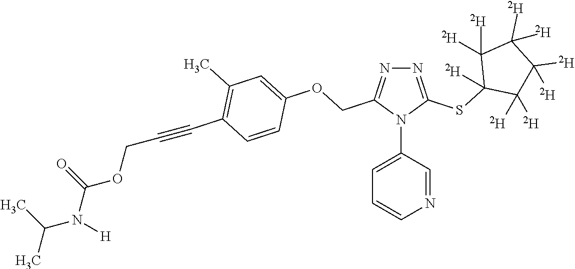 [SMILES: [2H]C1([2H])C([2H])([2H])C([2H])([2H])C([2H])(Sc2nnc(COc3ccc(C#CCOC(=O)NC(C)C)c(C)c3)n2-c2cccnc2)C1([2H])[2H]] |
| 24 | 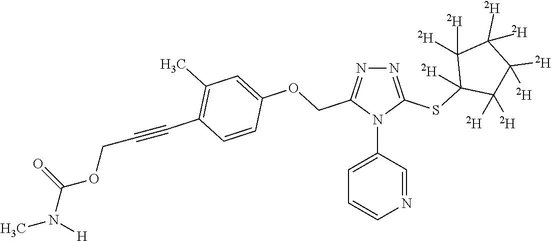 [SMILES: [2H]C1([2H])C([2H])([2H])C([2H])([2H])C([2H])(Sc2nnc(COc3ccc(C#CCOC(=O)NC)c(C)c3)n2-c2cccnc2)C1([2H])[2H]] |
| 25 | 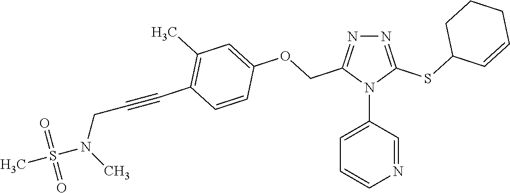 [SMILES: Cc1cc(OCc2nnc(SC3C=CCCC3)n2-c2cccnc2)ccc1C#CCN(C)S(C)(=O)=O] |
| 26 | 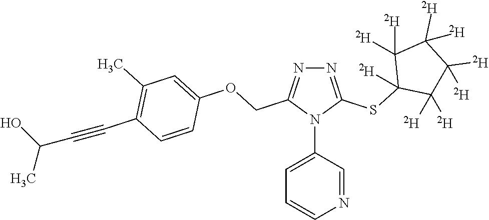 [SMILES: [2H]C1([2H])C([2H])([2H])C([2H])([2H])C([2H])(Sc2nnc(COc3ccc(C#CC(C)O)c(C)c3)n2-c2cccnc2)C1([2H])[2H]] |
| 27 | 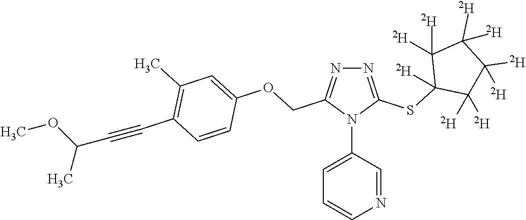 [SMILES: [2H]C1([2H])C([2H])([2H])C([2H])([2H])C([2H])(Sc2nnc(COc3ccc(C#CC(C)OC)c(C)c3)n2-c2cccnc2)C1([2H])[2H]] |
| 28 | 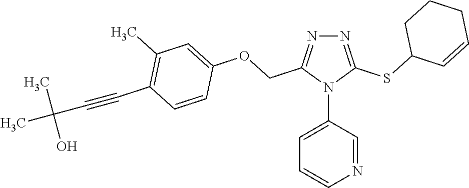 [SMILES: Cc1cc(OCc2nnc(SC3C=CCCC3)n2-c2cccnc2)ccc1C#CC(C)(C)O] |
| 29 | 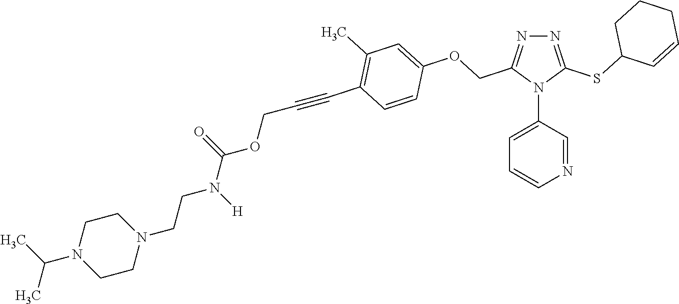 [SMILES: Cc1cc(OCc2nnc(SC3C=CCCC3)n2-c2cccnc2)ccc1C#CCOC(=O)NCCN1CCN(C(C)C)CC1] |
| 30 | 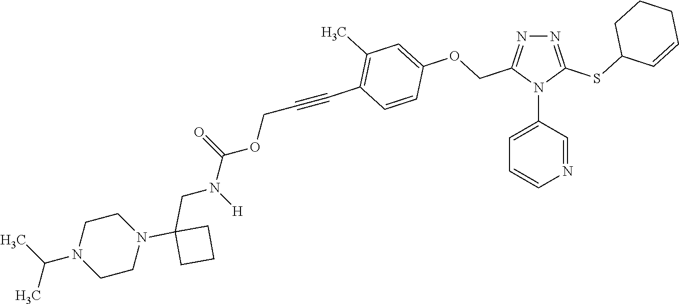 [SMILES: Cc1cc(OCc2nnc(SC3C=CCCC3)n2-c2cccnc2)ccc1C#CCOC(=O)NCC1(N2CCN(C(C)C)CC2)CCC1] |
| 31 | 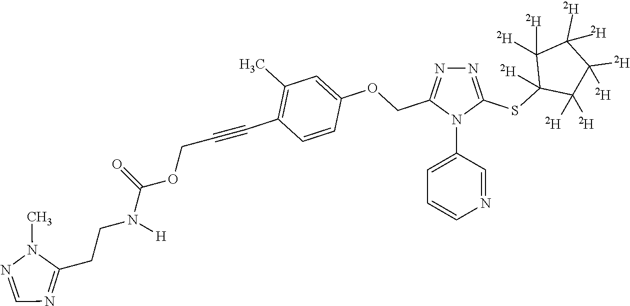 [SMILES: [2H]C1([2H])C([2H])([2H])C([2H])([2H])C([2H])(Sc2nnc(COc3ccc(C#CCOC(=O)NCCc4ncnn4C)c(C)c3)n2-c2cccnc2)C1([2H])[2H]] |
| 32 | 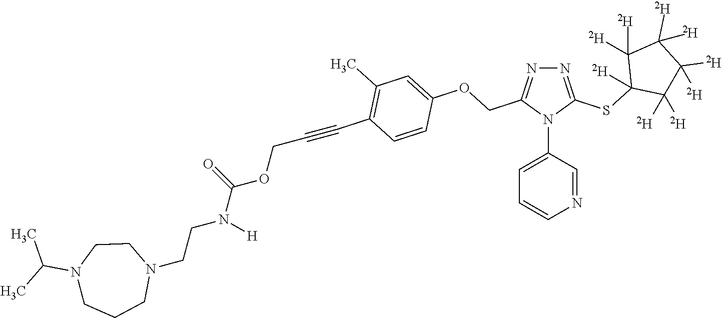 [SMILES: [2H]C1([2H])C([2H])([2H])C([2H])([2H])C([2H])(Sc2nnc(COc3ccc(C#CCOC(=O)NCCN4CCCN(C(C)C)CC4)c(C)c3)n2-c2cccnc2)C1([2H])[2H]] |
| 33 | 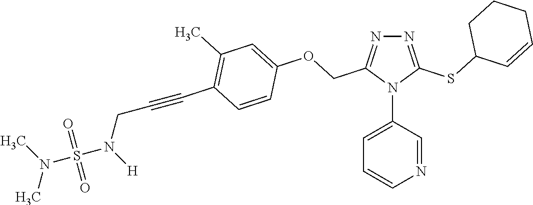 [SMILES: Cc1cc(OCc2nnc(SC3C=CCCC3)n2-c2cccnc2)ccc1C#CCNS(=O)(=O)N(C)C] |
| 34 | 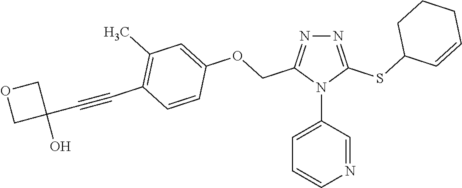 [SMILES: Cc1cc(OCc2nnc(SC3C=CCCC3)n2-c2cccnc2)ccc1C#CC1(O)COC1] |
| 35 | 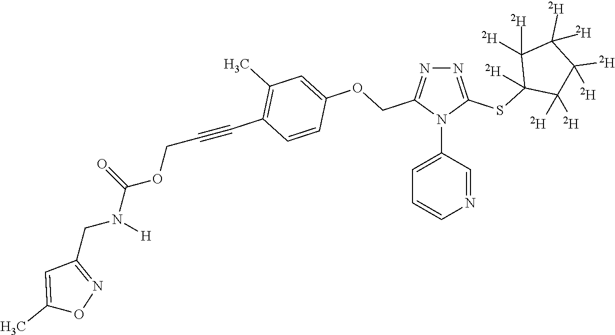 [SMILES: [2H]C1([2H])C([2H])([2H])C([2H])([2H])C([2H])(Sc2nnc(COc3ccc(C#CCOC(=O)NCc4cc(C)on4)c(C)c3)n2-c2cccnc2)C1([2H])[2H]] |
| 36 | 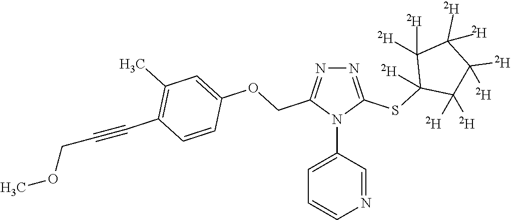 [SMILES: [2H]C1([2H])C([2H])([2H])C([2H])([2H])C([2H])(Sc2nnc(COc3ccc(C#CCOC)c(C)c3)n2-c2cccnc2)C1([2H])[2H]] |
| 37 | 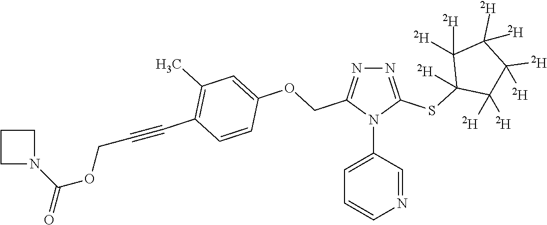 [SMILES: [2H]C1([2H])C([2H])([2H])C([2H])([2H])C([2H])(Sc2nnc(COc3ccc(C#CCOC(=O)N4CCC4)c(C)c3)n2-c2cccnc2)C1([2H])[2H]] |
| 38 | 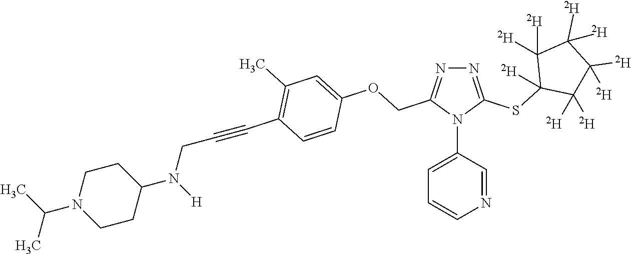 [SMILES: [2H]C1([2H])C([2H])([2H])C([2H])([2H])C([2H])(Sc2nnc(COc3ccc(C#CCNC4CCN(C(C)C)CC4)c(C)c3)n2-c2cccnc2)C1([2H])[2H]] |
| 39 | 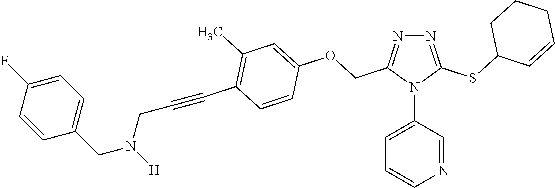 [SMILES: Cc1cc(OCc2nnc(SC3C=CCCC3)n2-c2cccnc2)ccc1C#CCNCc1ccc(F)cc1] |
| 40 | 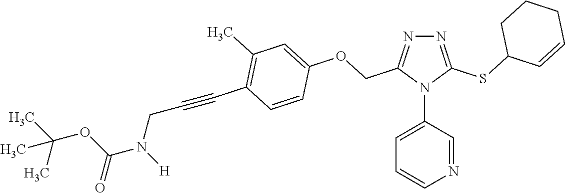 [SMILES: Cc1cc(OCc2nnc(SC3C=CCCC3)n2-c2cccnc2)ccc1C#CCNC(=O)OC(C)(C)C] |
| 41 | 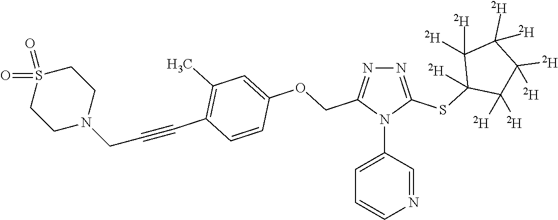 [SMILES: [2H]C1([2H])C([2H])([2H])C([2H])([2H])C([2H])(Sc2nnc(COc3ccc(C#CCN4CCS(=O)(=O)CC4)c(C)c3)n2-c2cccnc2)C1([2H])[2H]] |
| 42 | 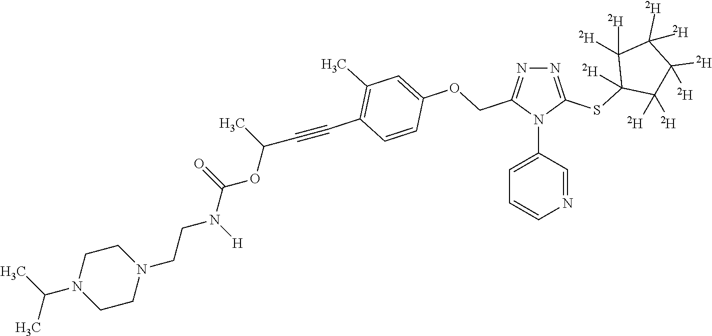 [SMILES: [2H]C1([2H])C([2H])([2H])C([2H])([2H])C([2H])(Sc2nnc(COc3ccc(C#CC(C)OC(=O)NCCN4CCN(C(C)C)CC4)c(C)c3)n2-c2cccnc2)C1([2H])[2H]] |
| 43 | 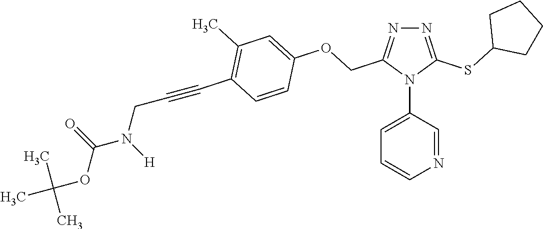 [SMILES: Cc1cc(OCc2nnc(SC3CCCC3)n2-c2cccnc2)ccc1C#CCNC(=O)OC(C)(C)C] |
| 44 | 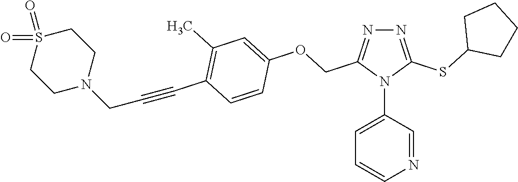 [SMILES: Cc1cc(OCc2nnc(SC3CCCC3)n2-c2cccnc2)ccc1C#CCN1CCS(=O)(=O)CC1] |
| 45 | 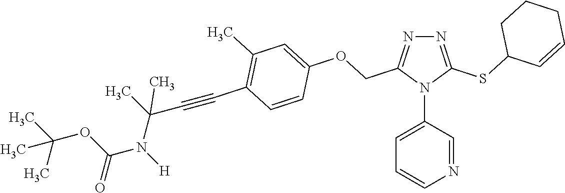 [SMILES: Cc1cc(OCc2nnc(SC3C=CCCC3)n2-c2cccnc2)ccc1C#CC(C)(C)NC(=O)OC(C)(C)C] |
| 46 | 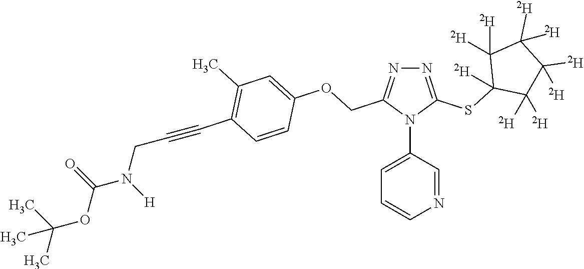 [SMILES: [2H]C1([2H])C([2H])([2H])C([2H])([2H])C([2H])(Sc2nnc(COc3ccc(C#CCNC(=O)OC(C)(C)C)c(C)c3)n2-c2cccnc2)C1([2H])[2H]] |
| 47 | 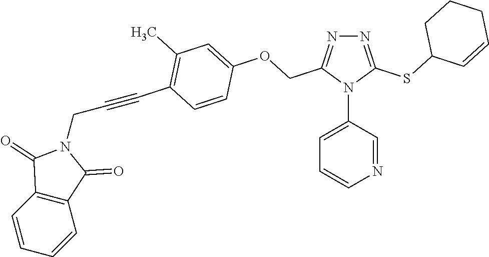 [SMILES: Cc1cc(OCc2nnc(SC3C=CCCC3)n2-c2cccnc2)ccc1C#CCN1C(=O)c2ccccc2C1=O] |
| 48 | 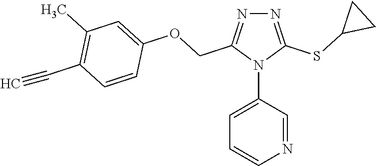 [SMILES: C#Cc1ccc(OCc2nnc(SC3CC3)n2-c2cccnc2)cc1C] |
| 49 | 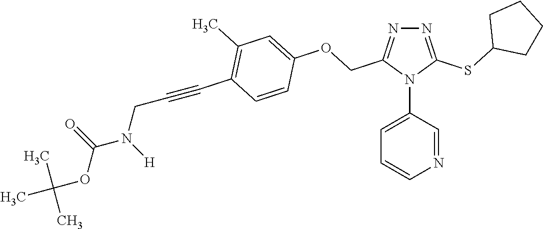 [SMILES: Cc1cc(OCc2nnc(SC3CCCC3)n2-c2cccnc2)ccc1C#CCNC(=O)OC(C)(C)C] |
| 50 | 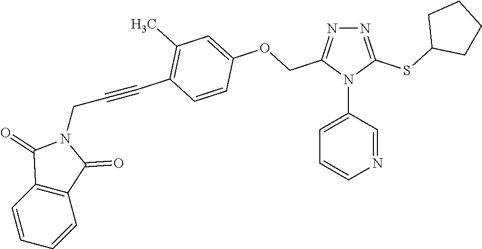 [SMILES: Cc1cc(OCc2nnc(SC3CCCC3)n2-c2cccnc2)ccc1C#CCN1C(=O)c2ccccc2C1=O] |
| 51 | 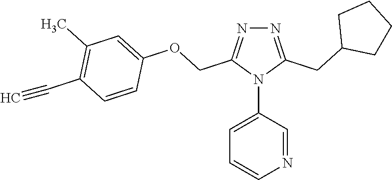 [SMILES: C#Cc1ccc(OCc2nnc(CC3CCCC3)n2-c2cccnc2)cc1C] |
| 52 | 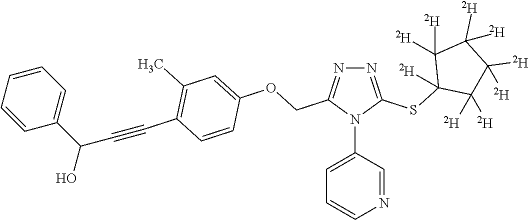 [SMILES: [2H]C1([2H])C([2H])([2H])C([2H])([2H])C([2H])(Sc2nnc(COc3ccc(C#CC(O)c4ccccc4)c(C)c3)n2-c2cccnc2)C1([2H])[2H]] |
| 53 | 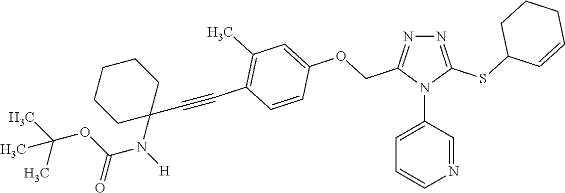 [SMILES: Cc1cc(OCc2nnc(SC3C=CCCC3)n2-c2cccnc2)ccc1C#CC1(NC(=O)OC(C)(C)C)CCCCC1] |
| 54 | 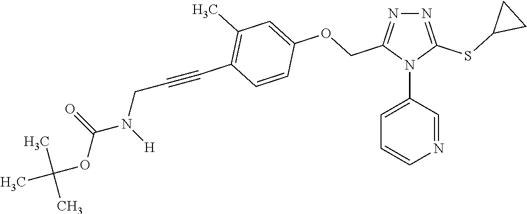 [SMILES: Cc1cc(OCc2nnc(SC3CC3)n2-c2cccnc2)ccc1C#CCNC(=O)OC(C)(C)C] |
| 55 | 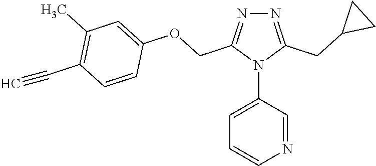 [SMILES: C#Cc1ccc(OCc2nnc(CC3CC3)n2-c2cccnc2)cc1C] |
| 56 | 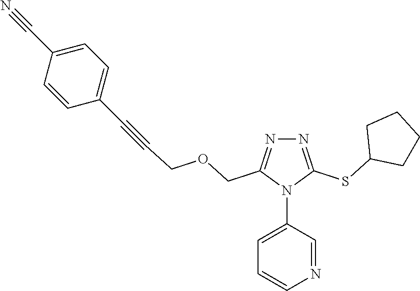 [SMILES: N#Cc1ccc(C#CCOCc2nnc(SC3CCCC3)n2-c2cccnc2)cc1] |
| 57 | 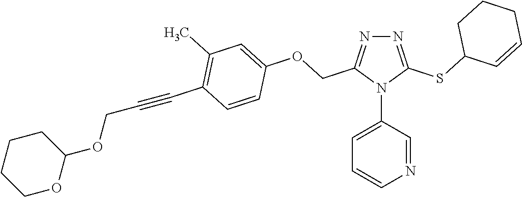 [SMILES: Cc1cc(OCc2nnc(SC3C=CCCC3)n2-c2cccnc2)ccc1C#CCOC1CCCCO1] |
| 58 | 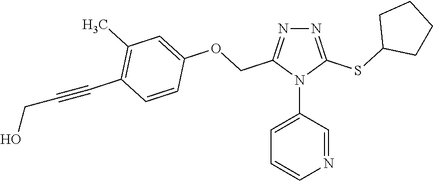 [SMILES: Cc1cc(OCc2nnc(SC3CCCC3)n2-c2cccnc2)ccc1C#CCO] |
| 59 | 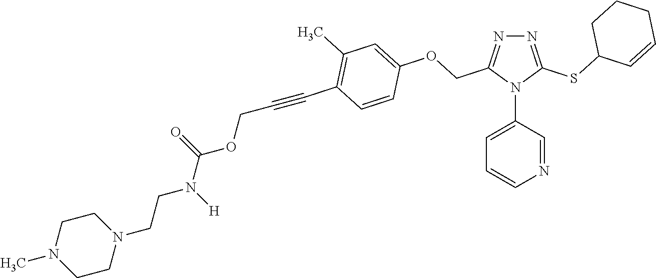 [SMILES: Cc1cc(OCc2nnc(SC3C=CCCC3)n2-c2cccnc2)ccc1C#CCOC(=O)NCCN1CCN(C)CC1] |
| 60 | 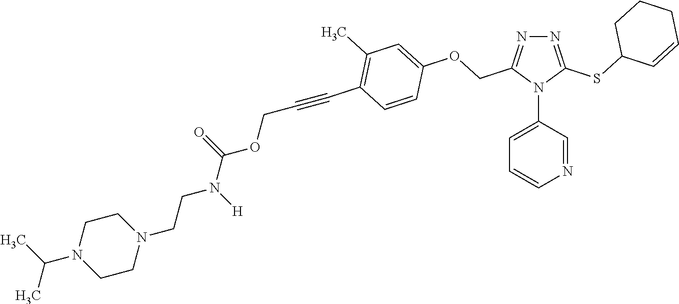 [SMILES: Cc1cc(OCc2nnc(SC3C=CCCC3)n2-c2cccnc2)ccc1C#CCOC(=O)NCCN1CCN(C(C)C)CC1] |
| 61 | 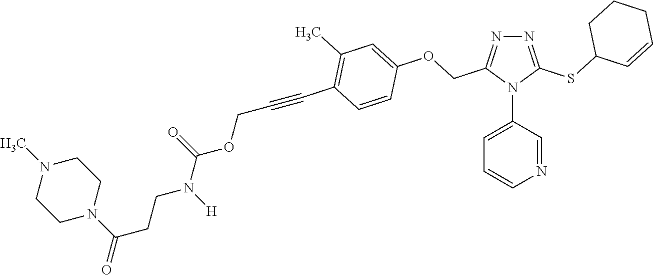 [SMILES: Cc1cc(OCc2nnc(SC3C=CCCC3)n2-c2cccnc2)ccc1C#CCOC(=O)NCCC(=O)N1CCN(C)CC1] |
| 62 | 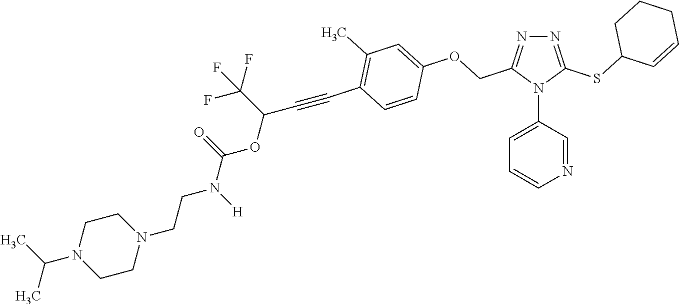 [SMILES: Cc1cc(OCc2nnc(SC3C=CCCC3)n2-c2cccnc2)ccc1C#CC(OC(=O)NCCN1CCN(C(C)C)CC1)C(F)(F)F] |
| 63 | 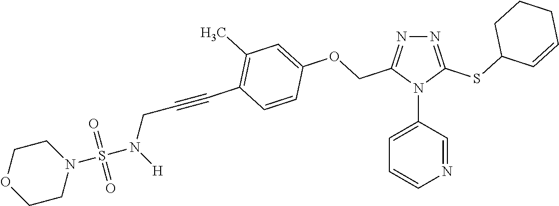 [SMILES: Cc1cc(OCc2nnc(SC3C=CCCC3)n2-c2cccnc2)ccc1C#CCNS(=O)(=O)N1CCOCC1] |
| 64 | 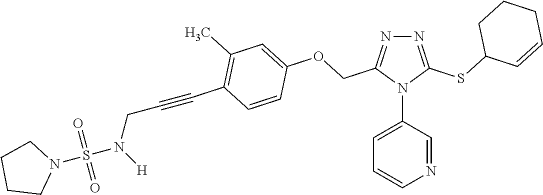 [SMILES: Cc1cc(OCc2nnc(SC3C=CCCC3)n2-c2cccnc2)ccc1C#CCNS(=O)(=O)N1CCCC1] |
| 65 | 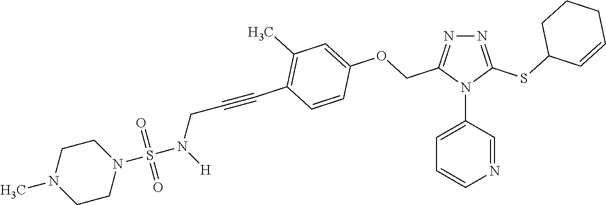 [SMILES: Cc1cc(OCc2nnc(SC3C=CCCC3)n2-c2cccnc2)ccc1C#CCNS(=O)(=O)N1CCN(C)CC1] |
| 66 | 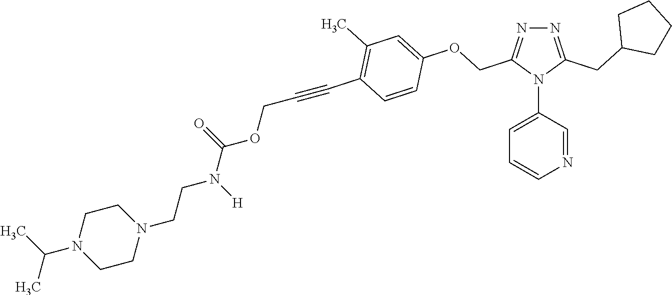 [SMILES: Cc1cc(OCc2nnc(CC3CCCC3)n2-c2cccnc2)ccc1C#CCOC(=O)NCCN1CCN(C(C)C)CC1] |
| 67 | 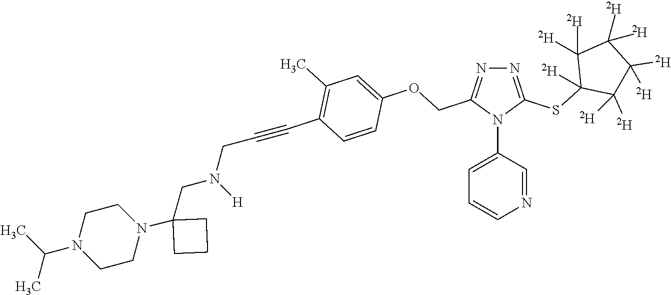 [SMILES: [2H]C1([2H])C([2H])([2H])C([2H])([2H])C([2H])(Sc2nnc(COc3ccc(C#CCNCC4(N5CCN(C(C)C)CC5)CCC4)c(C)c3)n2-c2cccnc2)C1([2H])[2H]] |
| 68 | 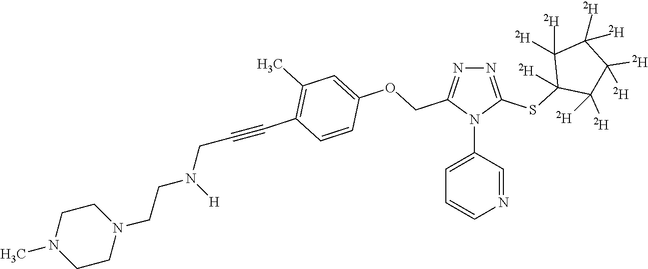 [SMILES: [2H]C1([2H])C([2H])([2H])C([2H])([2H])C([2H])(Sc2nnc(COc3ccc(C#CCNCCN4CCN(C)CC4)c(C)c3)n2-c2cccnc2)C1([2H])[2H]] |
| 69 | 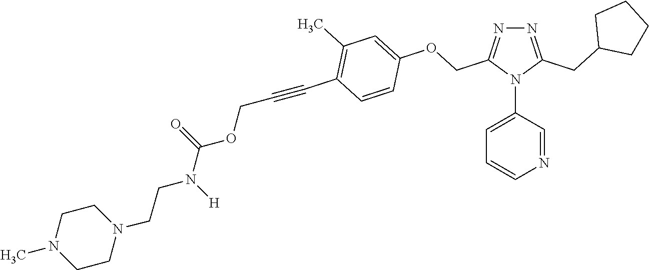 [SMILES: Cc1cc(OCc2nnc(CC3CCCC3)n2-c2cccnc2)ccc1C#CCOC(=O)NCCN1CCN(C)CC1] |
| 70 | 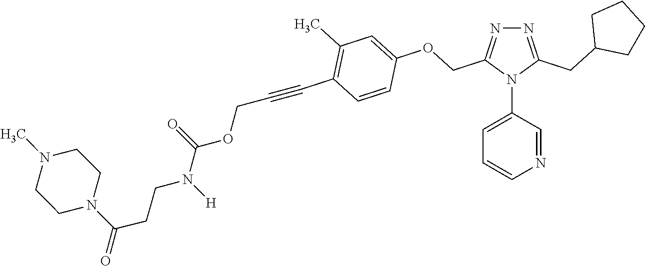 [SMILES: Cc1cc(OCc2nnc(CC3CCCC3)n2-c2cccnc2)ccc1C#CCOC(=O)NCCC(=O)N1CCN(C)CC1] |
| 71 | 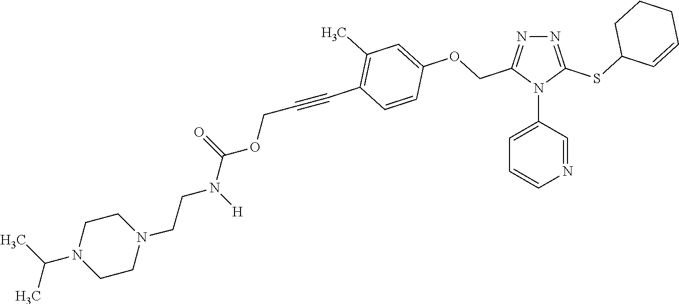 [SMILES: Cc1cc(OCc2nnc(SC3C=CCCC3)n2-c2cccnc2)ccc1C#CCOC(=O)NCCN1CCN(C(C)C)CC1] |
| 72 | 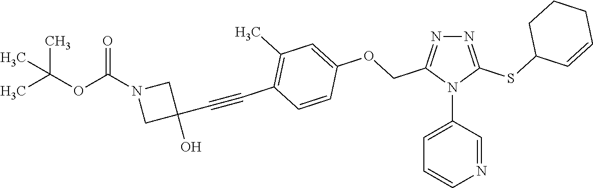 [SMILES: Cc1cc(OCc2nnc(SC3C=CCCC3)n2-c2cccnc2)ccc1C#CC1(O)CN(C(=O)OC(C)(C)C)C1] |
| 73 | 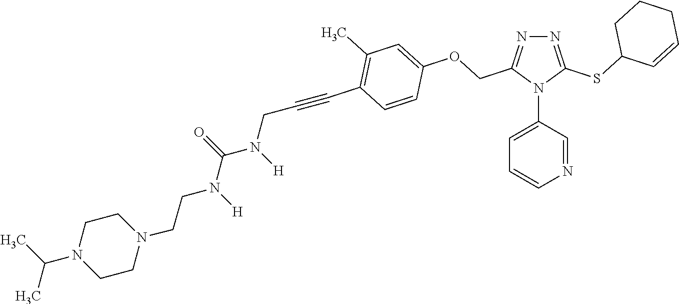 [SMILES: Cc1cc(OCc2nnc(SC3C=CCCC3)n2-c2cccnc2)ccc1C#CCNC(=O)NCCN1CCN(C(C)C)CC1] |
| 74 | 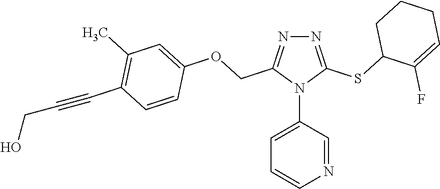 [SMILES: Cc1cc(OCc2nnc(SC3CCCC=C3F)n2-c2cccnc2)ccc1C#CCO] |
| 75 | 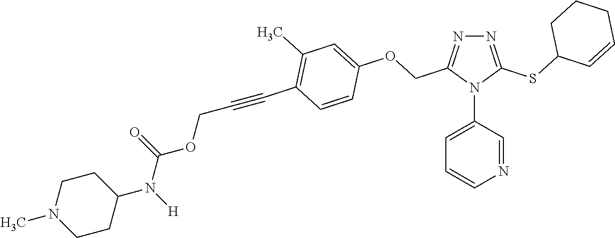 [SMILES: Cc1cc(OCc2nnc(SC3C=CCCC3)n2-c2cccnc2)ccc1C#CCOC(=O)NC1CCN(C)CC1] |
| 76 | 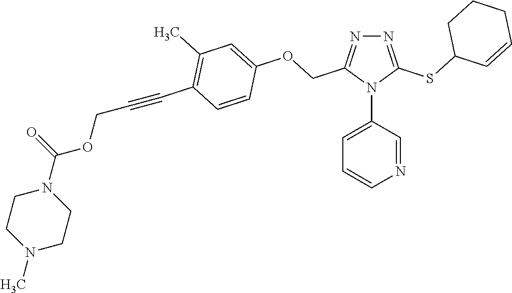 [SMILES: Cc1cc(OCc2nnc(SC3C=CCCC3)n2-c2cccnc2)ccc1C#CCOC(=O)N1CCN(C)CC1] |
| 77 | 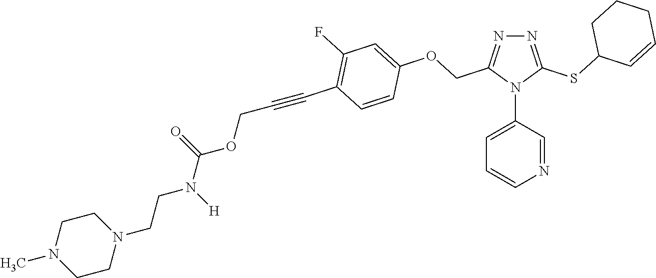 [SMILES: CN1CCN(CCNC(=O)OCC#Cc2ccc(OCc3nnc(SC4C=CCCC4)n3-c3cccnc3)cc2F)CC1] |
| 78 | 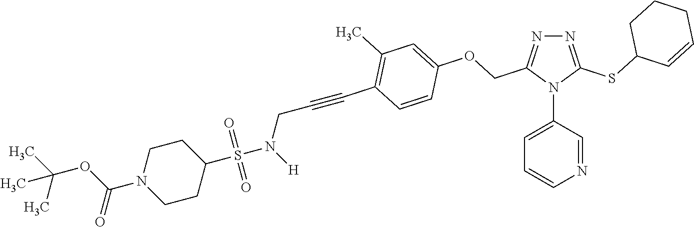 [SMILES: Cc1cc(OCc2nnc(SC3C=CCCC3)n2-c2cccnc2)ccc1C#CCNS(=O)(=O)C1CCN(C(=O)OC(C)(C)C)CC1] |
| 79 | 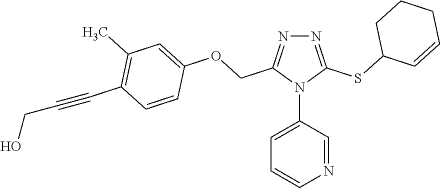 [SMILES: Cc1cc(OCc2nnc(SC3C=CCCC3)n2-c2cccnc2)ccc1C#CCO] |
| 80 | 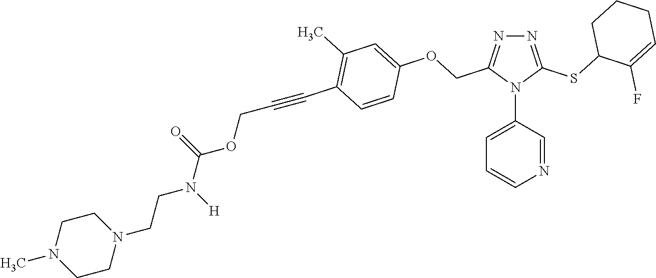 [SMILES: Cc1cc(OCc2nnc(SC3CCCC=C3F)n2-c2cccnc2)ccc1C#CCOC(=O)NCCN1CCN(C)CC1] |
| 81 | 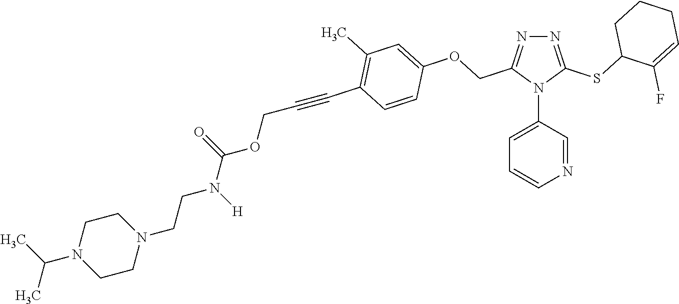 [SMILES: Cc1cc(OCc2nnc(SC3CCCC=C3F)n2-c2cccnc2)ccc1C#CCOC(=O)NCCN1CCN(C(C)C)CC1] |
| 82 | 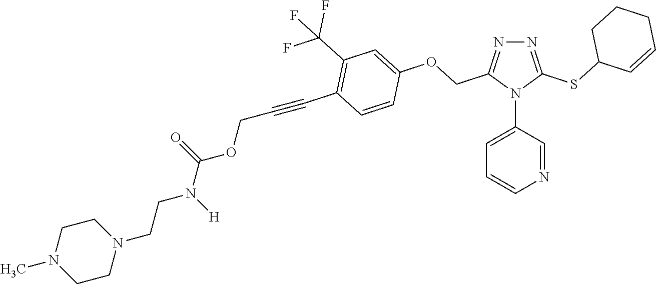 [SMILES: CN1CCN(CCNC(=O)OCC#Cc2ccc(OCc3nnc(SC4C=CCCC4)n3-c3cccnc3)cc2C(F)(F)F)CC1] |
| 83 | 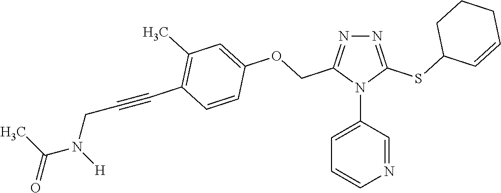 [SMILES: CC(=O)NCC#Cc1ccc(OCc2nnc(SC3C=CCCC3)n2-c2cccnc2)cc1C] |
| 84 | 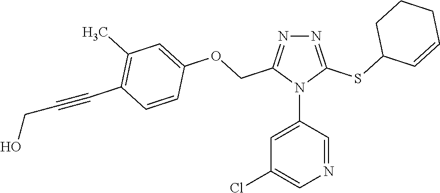 [SMILES: Cc1cc(OCc2nnc(SC3C=CCCC3)n2-c2cncc(Cl)c2)ccc1C#CCO] |
| 85 | 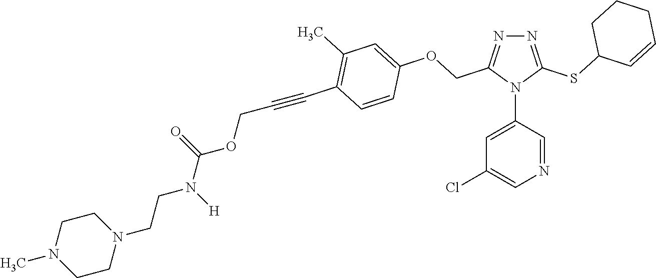 [SMILES: Cc1cc(OCc2nnc(SC3C=CCCC3)n2-c2cncc(Cl)c2)ccc1C#CCOC(=O)NCCN1CCN(C)CC1] |
| 86 | 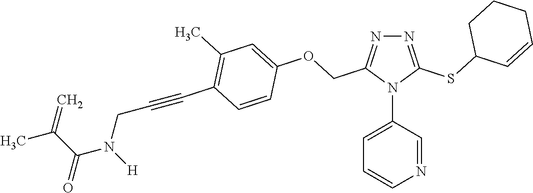 [SMILES: C=C(C)C(=O)NCC#Cc1ccc(OCc2nnc(SC3C=CCCC3)n2-c2cccnc2)cc1C] |
| 87 | 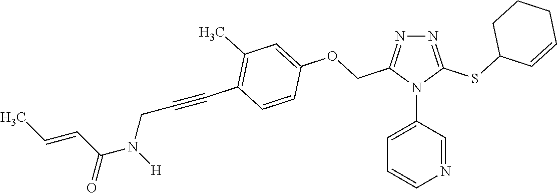 [SMILES: C/C=C/C(=O)NCC#Cc1ccc(OCc2nnc(SC3C=CCCC3)n2-c2cccnc2)cc1C] |
| 88 | 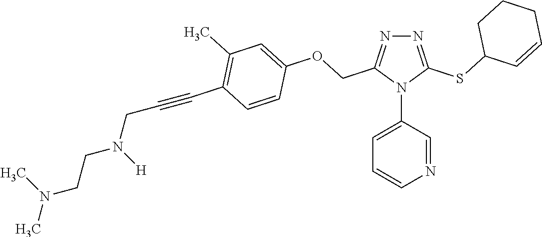 [SMILES: Cc1cc(OCc2nnc(SC3C=CCCC3)n2-c2cccnc2)ccc1C#CCNCCN(C)C] |
| 89 | 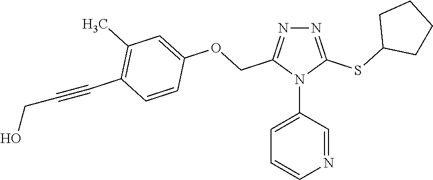 [SMILES: Cc1cc(OCc2nnc(SC3CCCC3)n2-c2cccnc2)ccc1C#CCO] |
| 90 | 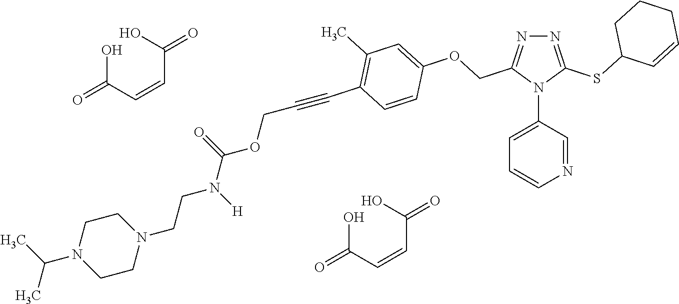 [SMILES: Cc1cc(OCc2nnc(SC3C=CCCC3)n2-c2cccnc2)ccc1C#CCOC(=O)NCCN1CCN(C(C)C)CC1 ; O=C(O)/C=C\C(=O)O ; O=C(O)/C=C\C(=O)O] |
| 91 | 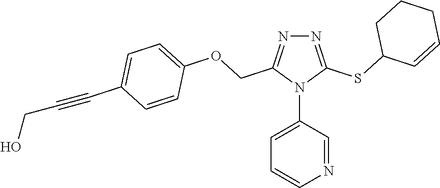 [SMILES: OCC#Cc1ccc(OCc2nnc(SC3C=CCCC3)n2-c2cccnc2)cc1] |
| 92 | 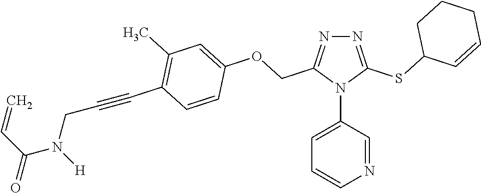 [SMILES: C=CC(=O)NCC#Cc1ccc(OCc2nnc(SC3C=CCCC3)n2-c2cccnc2)cc1C] |
| 93 | 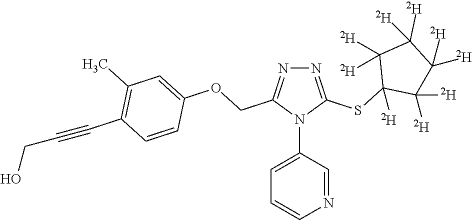 [SMILES: [2H]C1([2H])C([2H])([2H])C([2H])([2H])C([2H])(Sc2nnc(COc3ccc(C#CCO)c(C)c3)n2-c2cccnc2)C1([2H])[2H]] |
| 94 | 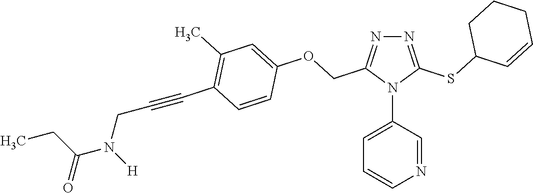 [SMILES: CCC(=O)NCC#Cc1ccc(OCc2nnc(SC3C=CCCC3)n2-c2cccnc2)cc1C] |
| 95 | 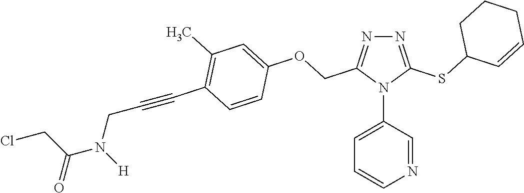 [SMILES: Cc1cc(OCc2nnc(SC3C=CCCC3)n2-c2cccnc2)ccc1C#CCNC(=O)CCl] |
| 96 | 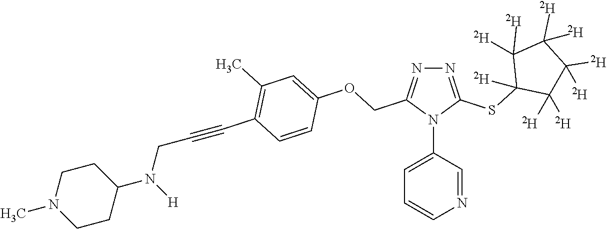 [SMILES: [2H]C1([2H])C([2H])([2H])C([2H])([2H])C([2H])(Sc2nnc(COc3ccc(C#CCNC4CCN(C)CC4)c(C)c3)n2-c2cccnc2)C1([2H])[2H]] |
| 97 | 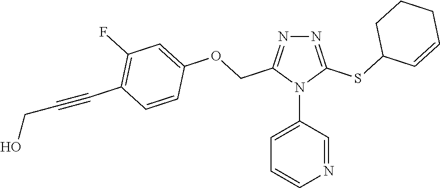 [SMILES: OCC#Cc1ccc(OCc2nnc(SC3C=CCCC3)n2-c2cccnc2)cc1F] |
| 98 | 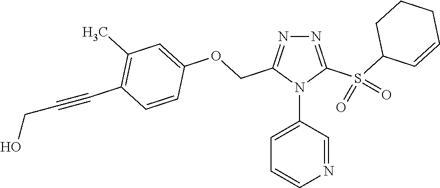 [SMILES: Cc1cc(OCc2nnc(S(=O)(=O)C3C=CCCC3)n2-c2cccnc2)ccc1C#CCO] |
| 99 | 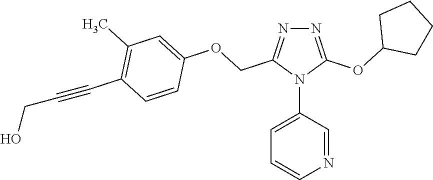 [SMILES: Cc1cc(OCc2nnc(OC3CCCC3)n2-c2cccnc2)ccc1C#CCO] |
| 100 | 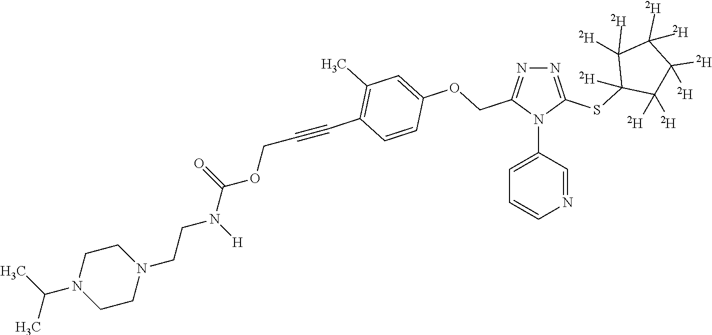 [SMILES: [2H]C1([2H])C([2H])([2H])C([2H])([2H])C([2H])(Sc2nnc(COc3ccc(C#CCOC(=O)NCCN4CCN(C(C)C)CC4)c(C)c3)n2-c2cccnc2)C1([2H])[2H]] |
| 101 | 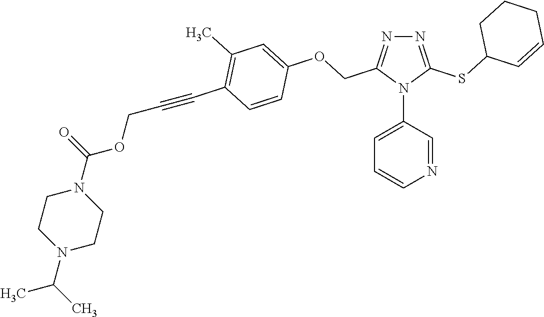 [SMILES: Cc1cc(OCc2nnc(SC3C=CCCC3)n2-c2cccnc2)ccc1C#CCOC(=O)N1CCN(C(C)C)CC1] |
| 102 | 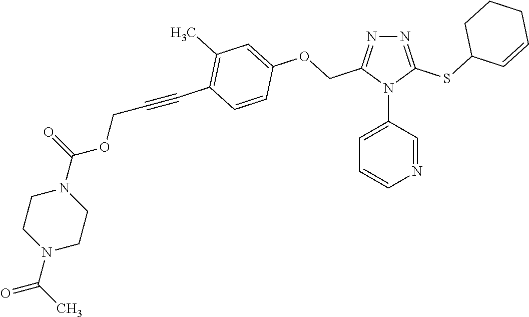 [SMILES: CC(=O)N1CCN(C(=O)OCC#Cc2ccc(OCc3nnc(SC4C=CCCC4)n3-c3cccnc3)cc2C)CC1] |
| 103 | 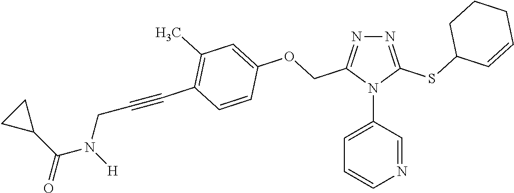 [SMILES: Cc1cc(OCc2nnc(SC3C=CCCC3)n2-c2cccnc2)ccc1C#CCNC(=O)C1CC1] |
| 104 | 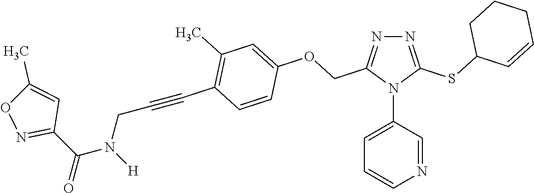 [SMILES: Cc1cc(C(=O)NCC#Cc2ccc(OCc3nnc(SC4C=CCCC4)n3-c3cccnc3)cc2C)no1] |
| 105 | 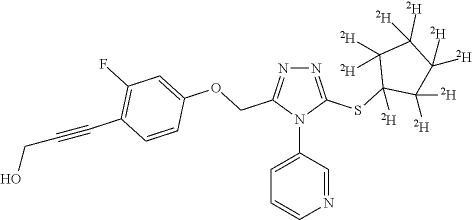 [SMILES: [2H]C1([2H])C([2H])([2H])C([2H])([2H])C([2H])(Sc2nnc(COc3ccc(C#CCO)c(F)c3)n2-c2cccnc2)C1([2H])[2H]] |
| 106 | 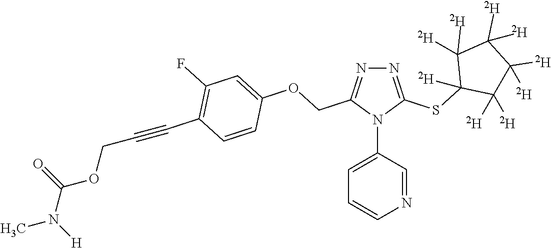 [SMILES: [2H]C1([2H])C([2H])([2H])C([2H])([2H])C([2H])(Sc2nnc(COc3ccc(C#CCOC(=O)NC)c(F)c3)n2-c2cccnc2)C1([2H])[2H]] |
| 107 | 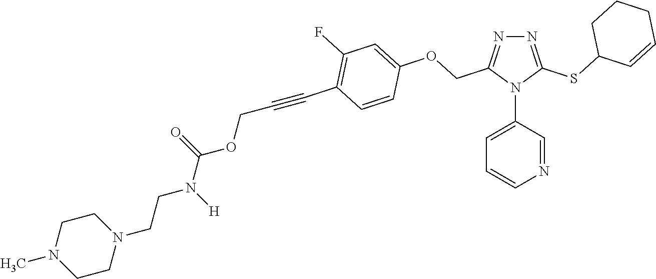 [SMILES: CN1CCN(CCNC(=O)OCC#Cc2ccc(OCc3nnc(SC4C=CCCC4)n3-c3cccnc3)cc2F)CC1] |
| 108 | 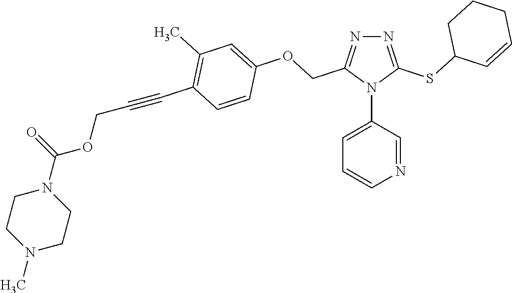 [SMILES: Cc1cc(OCc2nnc(SC3C=CCCC3)n2-c2cccnc2)ccc1C#CCOC(=O)N1CCN(C)CC1] |
| 109 | 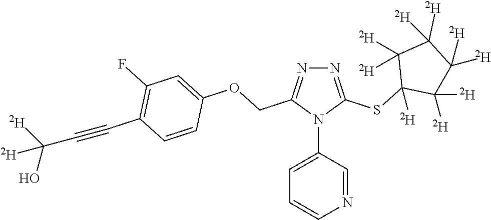 [SMILES: [2H]C([2H])(O)C#Cc1ccc(OCc2nnc(SC3([2H])C([2H])([2H])C([2H])([2H])C([2H])([2H])C3([2H])[2H])n2-c2cccnc2)cc1F] |
| 110 | 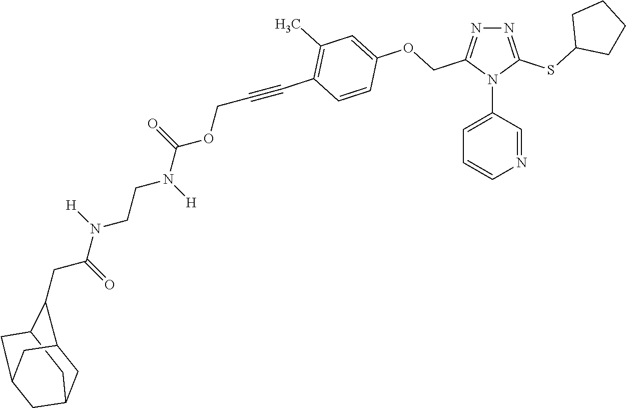 [SMILES: Cc1cc(OCc2nnc(SC3CCCC3)n2-c2cccnc2)ccc1C#CCOC(=O)NCCNC(=O)CC1C2CC3CC(C2)CC1C3] |
| 111 | 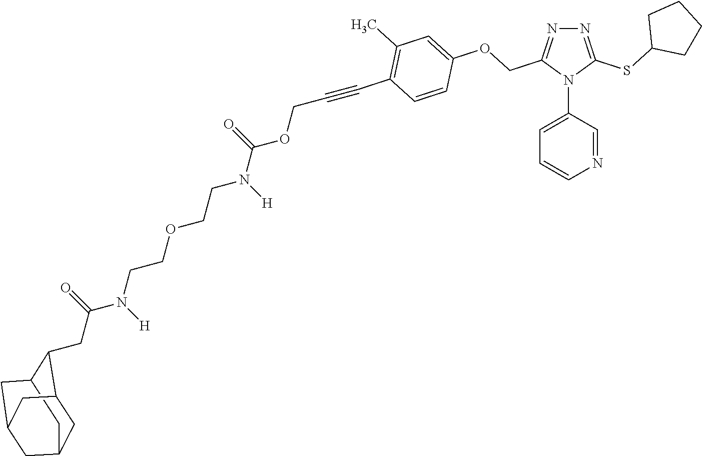 [SMILES: Cc1cc(OCc2nnc(SC3CCCC3)n2-c2cccnc2)ccc1C#CCOC(=O)NCCOCCNC(=O)CC1C2CC3CC(C2)CC1C3] |
| 112 | 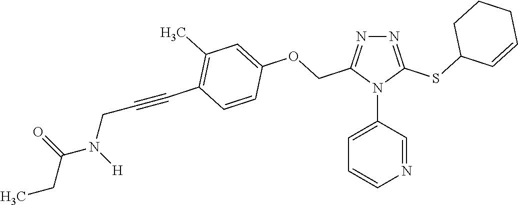 [SMILES: CCC(=O)NCC#Cc1ccc(OCc2nnc(SC3C=CCCC3)n2-c2cccnc2)cc1C] |
| 113 | 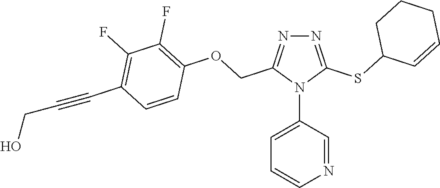 [SMILES: OCC#Cc1ccc(OCc2nnc(SC3C=CCCC3)n2-c2cccnc2)c(F)c1F] |
| 114 | 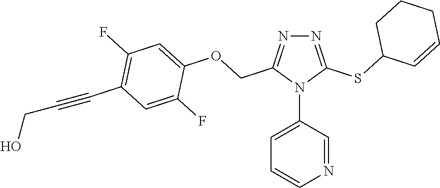 [SMILES: OCC#Cc1cc(F)c(OCc2nnc(SC3C=CCCC3)n2-c2cccnc2)cc1F] |
| 115 | 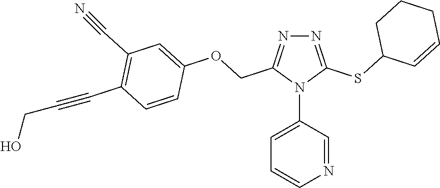 [SMILES: N#Cc1cc(OCc2nnc(SC3C=CCCC3)n2-c2cccnc2)ccc1C#CCO] |
| 116 | 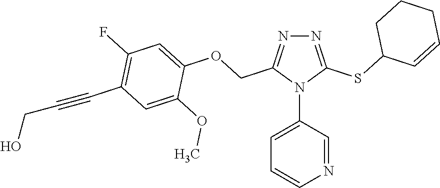 [SMILES: COc1cc(C#CCO)c(F)cc1OCc1nnc(SC2C=CCCC2)n1-c1cccnc1] |
| 117 | 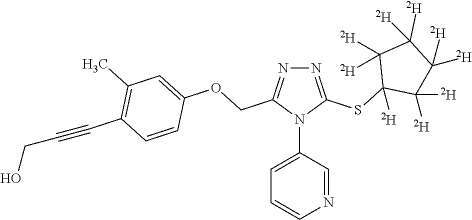 [SMILES: [2H]C1([2H])C([2H])([2H])C([2H])([2H])C([2H])(Sc2nnc(COc3ccc(C#CCO)c(C)c3)n2-c2cccnc2)C1([2H])[2H]] |
| 118 | 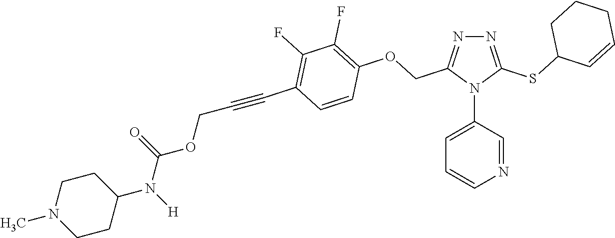 [SMILES: CN1CCC(NC(=O)OCC#Cc2ccc(OCc3nnc(SC4C=CCCC4)n3-c3cccnc3)c(F)c2F)CC1] |
| 119 | 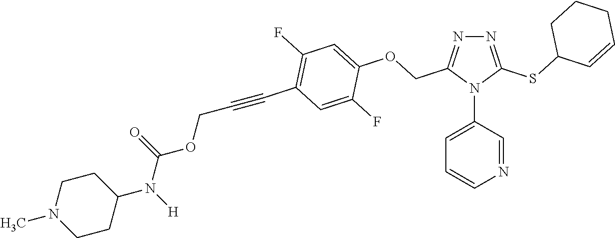 [SMILES: CN1CCC(NC(=O)OCC#Cc2cc(F)c(OCc3nnc(SC4C=CCCC4)n3-c3cccnc3)cc2F)CC1] |
| 120 | 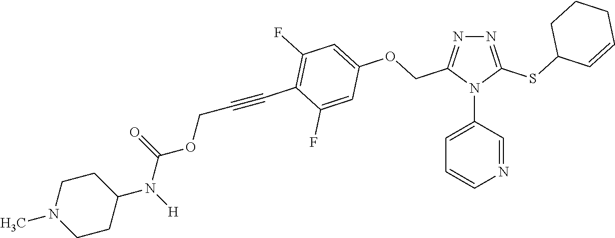 [SMILES: CN1CCC(NC(=O)OCC#Cc2c(F)cc(OCc3nnc(SC4C=CCCC4)n3-c3cccnc3)cc2F)CC1] |
| 121 | 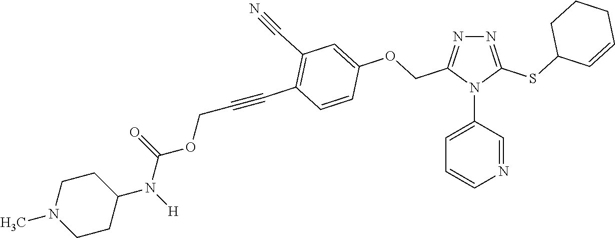 [SMILES: CN1CCC(NC(=O)OCC#Cc2ccc(OCc3nnc(SC4C=CCCC4)n3-c3cccnc3)cc2C#N)CC1] |
| 122 | 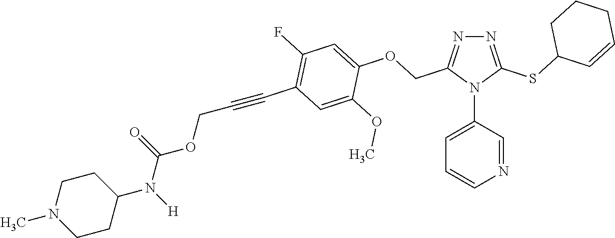 [SMILES: COc1cc(C#CCOC(=O)NC2CCN(C)CC2)c(F)cc1OCc1nnc(SC2C=CCCC2)n1-c1cccnc1] |
| 123 | 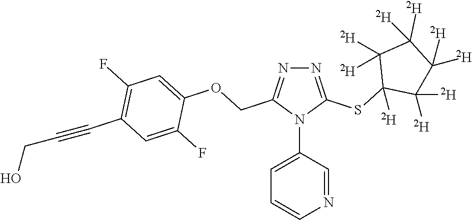 [SMILES: [2H]C1([2H])C([2H])([2H])C([2H])([2H])C([2H])(Sc2nnc(COc3cc(F)c(C#CCO)cc3F)n2-c2cccnc2)C1([2H])[2H]] |
| 124 | 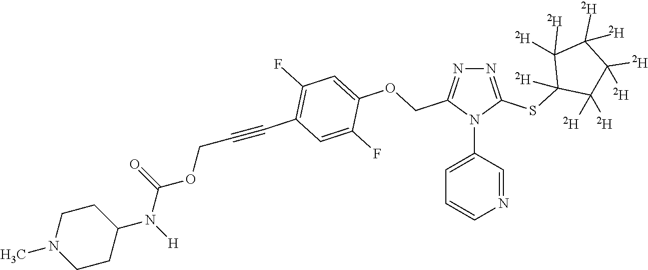 [SMILES: [2H]C1([2H])C([2H])([2H])C([2H])([2H])C([2H])(Sc2nnc(COc3cc(F)c(C#CCOC(=O)NC4CCN(C)CC4)cc3F)n2-c2cccnc2)C1([2H])[2H]] |
| 125 | 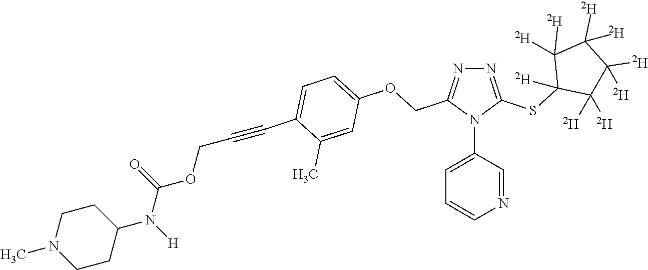 [SMILES: [2H]C1([2H])C([2H])([2H])C([2H])([2H])C([2H])(Sc2nnc(COc3ccc(C#CCOC(=O)NC4CCN(C)CC4)c(C)c3)n2-c2cccnc2)C1([2H])[2H]] |
| 126 | 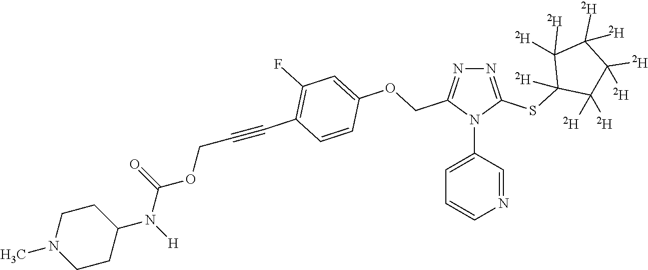 [SMILES: [2H]C1([2H])C([2H])([2H])C([2H])([2H])C([2H])(Sc2nnc(COc3ccc(C#CCOC(=O)NC4CCN(C)CC4)c(F)c3)n2-c2cccnc2)C1([2H])[2H]] |
| 127 | 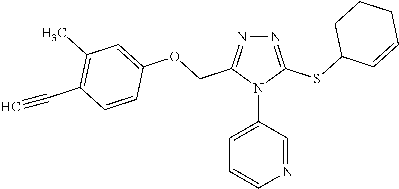 [SMILES: C#Cc1ccc(OCc2nnc(SC3C=CCCC3)n2-c2cccnc2)cc1C] |
| 128 | 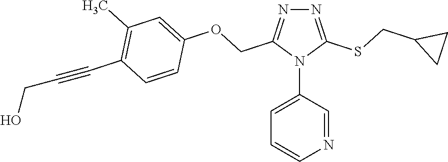 [SMILES: Cc1cc(OCc2nnc(SCC3CC3)n2-c2cccnc2)ccc1C#CCO] |
| 129 | 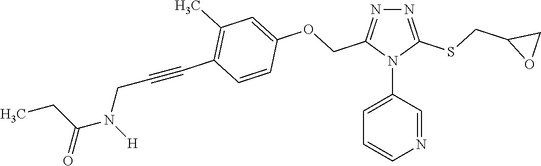 [SMILES: CCC(=O)NCC#Cc1ccc(OCc2nnc(SCC3CO3)n2-c2cccnc2)cc1C] |
| 130 | 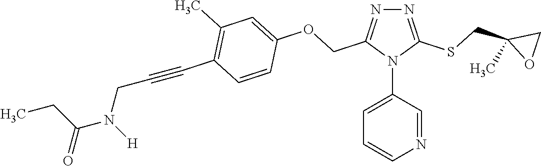 [SMILES: CCC(=O)NCC#Cc1ccc(OCc2nnc(SC[C@@]3(C)CO3)n2-c2cccnc2)cc1C] |
| 131 | 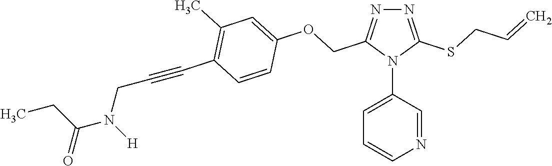 [SMILES: C=CCSc1nnc(COc2ccc(C#CCNC(=O)CC)c(C)c2)n1-c1cccnc1] |
| 132 | 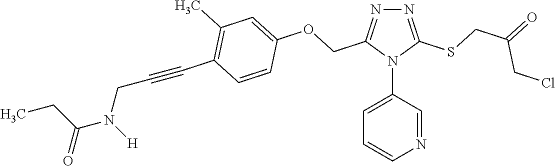 [SMILES: CCC(=O)NCC#Cc1ccc(OCc2nnc(SCC(=O)CCl)n2-c2cccnc2)cc1C] |
| 133 | 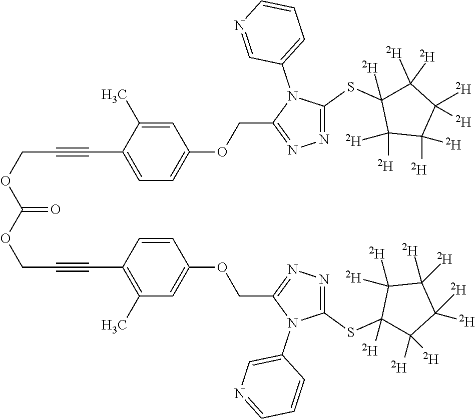 [SMILES: [2H]C1([2H])C([2H])([2H])C([2H])([2H])C([2H])(Sc2nnc(COc3ccc(C#CCOC(=O)OCC#Cc4ccc(OCc5nnc(SC6([2H])C([2H])([2H])C([2H])([2H])C([2H])([2H])C6([2H])[2H])n5-c5cccnc5)cc4C)c(C)c3)n2-c2cccnc2)C1([2H])[2H]] |
| 134 | 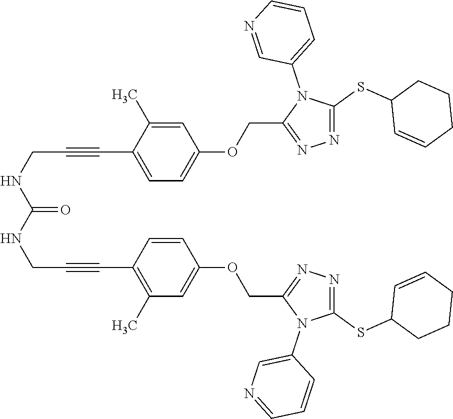 [SMILES: Cc1cc(OCc2nnc(SC3C=CCCC3)n2-c2cccnc2)ccc1C#CCNC(=O)NCC#Cc1ccc(OCc2nnc(SC3C=CCCC3)n2-c2cccnc2)cc1C] |

Treatment Methods and Uses

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition. The therapeutically effective amount may vary depending on the subject, and disease or condition being treated, the weight and age of the subject, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

In one embodiment, the present disclosure provides a method of using the compounds and compositions of the present disclosure to treat a multisystem degenerative disease in a patient in need thereof.

Non-limiting examples of multisystem degenerative disease include inclusion body myopathy, Paget's disease of bone, frontotemporal dementia, and amyotrophic lateral sclerosis (ALS).

Inclusion body myopathies, or hereditary inclusion body myopathies (HIBM), are a group of rare genetic disorders which have different symptoms. Generally, they are neuromuscular disorders characterized by muscle weakness developing in young adults. Hereditary inclusion body myopathies comprise both autosomal recessive and autosomal dominant muscle disorders that have a variable expression (phenotype) in individuals, but all share similar structural features in the muscles.

Paget's disease of bone (commonly known as Paget's disease or historically, osteitis deformans) is a condition involving cellular remodeling and deformity of one or more bones. The affected bones show signs of dysregulated bone remodeling at the microscopic level, specifically excessive bone breakdown and subsequent disorganized new bone formation. These structural changes cause the bone to weaken, which may result in deformity, pain, fracture, or arthritis of associated joints. The exact cause is unknown, although leading theories indicate both genetic and acquired factors (see causes). Paget's disease may affect any one or multiple bones of the body (most commonly pelvis, femur, and lumbar vertebrae, and skull), but never the entire skeleton, and does not spread from bone to bone. Rarely, a bone affected by Paget's disease can transform into a malignant bone cancer.

Frontotemporal dementias (FTD) encompasses six types of dementia involving the frontal or temporal lobes. They are: behavioral variant of FTD, semantic variant primary progressive aphasia, nonfluent agrammatic variant primary progressive aphasia, corticobasal syndrome, progressive supranuclear palsy, and FTD associated with motor neuron disease.

Amyotrophic lateral sclerosis (ALS), also known as motor neurone disease (MND), or Lou Gehrig's disease, is a specific disease which causes the death of neurons controlling voluntary muscles. Some also use the term motor neuron disease for a group of conditions of which ALS is the most common. ALS is characterized by stiff muscles, muscle twitching, and gradually worsening weakness due to muscles decreasing in size. This results in difficulty speaking, swallowing, and eventually breathing.

The presently disclosed compounds and compositions can also be used, in one embodiment, to treat a neurodegenerative disorder. Non-limiting examples of neurodegenerative disorder includes Alzheimer's disease (AD), Parkinson's disease (PD), Huntington's disease (HD), amyotrophic lateral sclerosis (ALS), and Batten disease.

Alzheimer's disease (AD), also referred to simply as Alzheimer's, is a chronic neurodegenerative disease that usually starts slowly and worsens over time. It is the cause of 60-70% of cases of dementia. The most common early symptom is difficulty in remembering recent events (short-term memory loss). As the disease advances, symptoms can include problems with language, disorientation (including easily getting lost), mood swings, loss of motivation, not managing self-care, and behavioral issues. As a person's condition declines, they often withdraw from family and society. Gradually, bodily functions are lost, ultimately leading to death. Although the speed of progression can vary, the typical life expectancy following diagnosis is three to nine years.

Parkinson's disease (PD) is a long-term degenerative disorder of the central nervous system that mainly affects the motor system. The symptoms generally come on slowly over time. Early in the disease, the most obvious are shaking, rigidity, slowness of movement, and difficulty with walking. Thinking and behavioral problems may also occur. Dementia becomes common in the advanced stages of the disease. Depression and anxiety are also common, occurring in more than a third of people with PD. Other symptoms include sensory, sleep, and emotional problems. The main motor symptoms are collectively called “parkinsonism”, or a “parkinsonian syndrome”.

Huntington's disease (HD), also known as Huntington's chorea, is an inherited disorder that results in death of brain cells. The earliest symptoms are often subtle problems with mood or mental abilities. A general lack of coordination and an unsteady gait often follow. As the disease advances, uncoordinated, jerky body movements become more apparent. Physical abilities gradually worsen until coordinated movement becomes difficult and the person is unable to talk. Mental abilities generally decline into dementia. The specific symptoms vary somewhat between people. Symptoms usually begin between 30 and 50 years of age, but can start at any age. The disease may develop earlier in life in each successive generation. About eight percent of cases start before the age of 20 years and typically present with symptoms more similar to Parkinson's disease.

Batten disease is a fatal disease of the nervous system that typically begins in childhood. Onset of symptoms is usually between 5 and 10 years of age. Often it is autosomal recessive. It is the most common form of a group of disorders called the neuronal ceroid lipofuscinoses (NCLs).

Various malignancies can also be treated with the presently disclosed compounds and compositions. In one embodiment, a method is provided for treating a hematological malignancy, or tumors of the hematopoietic and lymphoid tissues.

Tumors of the hematopoietic and lymphoid tissues or haematopoietic and lymphoid malignancies are tumors that affect the blood, bone marrow, lymph, and lymphatic system. As those elements are all intimately connected through both the circulatory system and the immune system, a disease affecting one will often affect the others as well, making myeloproliferation and lymphoproliferation (and thus the leukemias and the lymphomas) closely related and often overlapping problems.

Hematological malignancies may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

An example of myeloma is multiple myeloma (MM), a cancer of plasma cells, a type of white blood cell normally responsible for producing antibodies.

Acute myeloid leukemia (AML), also known as acute myelogenous leukemia or acute nonlymphocytic leukemia (ANLL), is a cancer of the myeloid line of blood cells, characterized by the rapid growth of abnormal white blood cells that accumulate in the bone marrow and interfere with the production of normal blood cells. AML is the most common acute leukemia affecting adults.

Myeloproliferative diseases (MPD), or the myeloproliferative neoplasms (MPNs), previously myeloproliferative diseases (MPDs), are a group of diseases of the bone marrow in which excess cells are produced. They are related to, and may evolve into, myelodysplastic syndrome and acute myeloid leukemia, although the myeloproliferative diseases on the whole have a much better prognosis than these conditions.

B-cell lymphomas are types of lymphoma affecting B cells. Lymphomas are “blood cancers” in the lymph nodes. B-cell lymphomas include both Hodgkin's lymphomas and most non-Hodgkin lymphomas.

Follicular lymphoma (FL)—Follicular lymphoma is a type of blood cancer. It is the most common of the indolent (slow-growing) non-Hodgkin's lymphomas, and the second-most-common form of non-Hodgkin's lymphomas overall. It is defined as a lymphoma of follicle center B-cells (centrocytes and centroblasts), which has at least a partially follicular pattern.

Chronic lymphoid leukemia (CLL), or B-cell chronic lymphocytic leukemia (B-CLL), also known as chronic lymphoid leukemia (CLL), is the most common type of leukemia (a type of cancer of the white blood cells) in adults. CLL affects B cell lymphocytes, which originate in the bone marrow, develop in the lymph nodes, and normally fight infection by producing antibodies.

CLL is a stage of small lymphocytic lymphoma (SLL), a type of B-cell lymphoma, which presents primarily in the lymph nodes. CLL and SLL are considered the same underlying disease, just with different appearances.

Diffuse large B-cell lymphoma (DLBCL or DLBL) is a cancer of B cells, a type of white blood cell responsible for producing antibodies. Diffuse large B-cell lymphoma encompasses a biologically and clinically diverse set of diseases, many of which cannot be separated from one another by well-defined and widely accepted criteria.

Leukemias include acute lymphoblastic leukemia (ALL), acute myelogenous leukemia (AML), chronic lymphocytic leukemia (CLL) small lymphocytic lymphoma (SLL) when leukemic cells are absent, chronic myelogenous leukemia (CML), acute monocytic leukemia (AMoL) and other leukemias.

Lymphomas can be Hodgkin's lymphomas (all four subtypes) and Non-Hodgkin's lymphomas (all subtypes).

The compounds are contemplated to be efficacious enough to treat hematological malignancy that is relapsed and refractory.

Solid tumors can also be treated. Cancer patients suitable for the treatment can be those having bladder cancer, liver cancer, colon cancer, rectal cancer, endometrial cancer, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, melanoma, prostate cancer or thyroid cancer. In some embodiments, the cancer comprises advanced solid tumor. In some embodiments, the cancer is relapsed and refractory.

The presently disclosed compounds can inhibit p97 with certain mutations that are known to be associated with certain patients of the above diseases and can cause resistance to treatment.

In some embodiments, the mutation is in the N domain of the p97 gene. An example is R155H.

In some embodiments, the mutation is in the N-D1 linker of the p97 gene. An example is L198W.

In some embodiments, the mutation is in the D1 domain of the p97 gene. An example is A232E. Two additional mutations in the D1 domains are K251A and E305Q.

In some embodiments, the mutation is in the D2 domain of the p97 gene. An example is K524A. Another example is E578Q.

### Pharmaceutical Compositions and Modes of Administration

Compounds provided herein are usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G.S. Banker & C.T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, tautomer, stereoisomer, mixture of stereoisomers, prodrug, or deuterated analog thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### Dosing

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound of Formula I may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Assay Method

This example tests the compounds of Table III in the assay procedure disclosed in Zhang, et al., “Altered cofactor regulation with disease-associated p97/VCP mutations,” Proc. Natl. Acad. Sci. USA, 112(14), E1705-E1714 (2015). The following Table IV provides relevant results. The compound number in Table IV correlates to the numbered compounds in Table III.

TABLE IV

| | P97 Biomolgreen; |
|---|---|
| | 200 μM ATP; |
| No. | IC₅₀ (μM) |
| 1 | 0.16 |
| 2 | 0.049 |
| 3 | 0.17 |
| 4 | 0.044 |
| 5 | 0.021 |
| 6 | 0.044 |
| 7 | 0.13 |
| 8 | 0.039 |
| 9 | 0.1 |
| 10 | 0.092 |
| 11 | 0.007 |
| 12 | 0.029 |
| 13 | 0.05 |
| 14 | 0.035 |
| 15 | 0.074 |
| 16 | 0.055 |
| 17 | 0.04 |
| 18 | 0.035 |
| 19 | 0.036 |
| 20 | 0.034 |
| 21 | 0.034 |
| 22 | 0.027 |
| 23 | 0.032 |
| 24 | 0.026 |
| 25 | 0.066 |
| 26 | 0.068 |
| 27 | 0.16 |
| 28 | 0.096 |
| 29 | 0.019 |
| 30 | 0.011 |
| 31 | 0.052 |
| 32 | 0.023 |
| 33 | 0.043 |
| 34 | 0.098 |
| 35 | 0.064 |
| 36 | 0.069 |
| 37 | 0.07 |
| 38 | 0.095 |
| 39 | 0.11 |
| 40 | 0.14 |
| 41 | 0.16 |
| 42 | 0.13 |
| 43 | 0.048 |
| 44 | 0.18 |
| 45 | 0.64 |
| 46 | 0.19 |
| 47 | 0.29 |
| 48 | 0.67 |
| 49 | 0.26 |
| 50 | 0.28 |
| 51 | 0.44 |
| 52 | 0.55 |
| 53 | 1.1 |
| 54 | 2 |
| 55 | 5.3 |
| 56 | >6.7 |
| 57 | 0.11 |
| 58 | 0.093 |
| 59 | 0.025 |
| 60 | 0.019 |
| 61 | 0.029 |
| 62 | 0.11 |
| 63 | 0.048 |
| 64 | 0.06 |
| 65 | 0.047 |
| 66 | 0.12 |
| 67 | 0.2 |
| 68 | 0.17 |
| 69 | 0.066 |
| 70 | 0.098 |
| 71 | 0.026 |
| 72 | 0.18 |
| 73 | 0.052 |
| 74 | 0.51 |
| 75 | 0.025 |
| 76 | 0.03 |
| 77 | 0.02 |
| 78 | 0.09 |
| 79 | 0.037 |
| 80 | 0.23 |
| 81 | 0.16 |
| 82 | 0.043 |
| 83 | 0.046 |
| 84 | 2.108 |
| 85 | 0.934 |
| 86 | 0.06 |
| 87 | 0.058 |
| 88 | 0.06 |
| 89 | 0.028 |
| 90 | 0.084 |
| 91 | 0.032 |
| 92 | 0.026 |
| 93 | 0.048 |
| 94 | 0.03 |
| 95 | 0.03 |
| 96 | 0.067 |
| 97 | 0.026 |
| 98 | 4.819 |
| 99 | 0.183 |
| 100 | 0.025 |
| 101 | 0.03 |
| 102 | 0.03 |
| 103 | 0.03 |
| 104 | 0.05 |
| 105 | 0.03 |
| 106 | 0.02 |
| 107 | 0.013 |
| 108 | 0.015 |
| 109 | 0.03 |
| 110 | 0.06 |
| 111 | 0.03 |
| 112 | 0.025 |
| 113 | 0.04 |
| 114 | 0.02 |
| 115 | 0.1 |
| 116 | 0.04 |
| 117 | 0.99 |
| 118 | 0.017 |
| 119 | 0.012 |
| 120 | 0.012 |
| 121 | 0.03 |
| 122 | 0.019 |
| 123 | 0.021 |
| 124 | 0.01 |
| 125 | 0.016 |
| 126 | 0.016 |
| 127 | 0.038 |
| 128 | 0.13 |
| 129 | Not active |
| 130 | Not active |
| 131 | 0.33 |
| 132 | 2.775 |
| 133 | 0.91 |
| 134 | 0.25 |

### Example 2: Assay Method

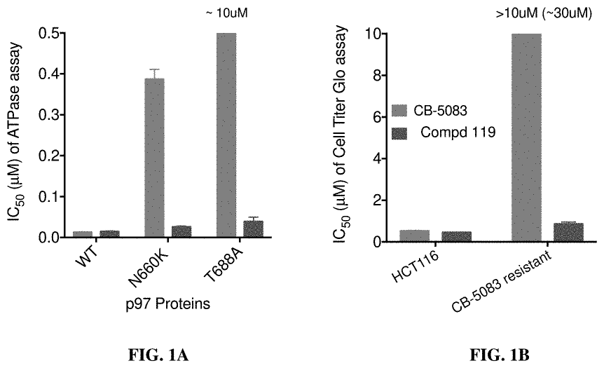

This example tests certain compounds of Table III in the assay procedure as follows. Cells were seeded on a 384-well solid white plate (5,000 cells/well). Cells were transfected with luciferase siRNA or p97 siRNA (10 nM) for 48 h or treated with compounds for the indicated amount of time. Caspase-3/7 Glo, caspase-6 Glo, caspase-8 Glo, or caspase-9 Glo (Promega) was added into each well and mixed by shaking at 500 rpm for 1 min. Luminescence signal was determined after incubation at room temperature for 1 h. Cellular viability was determined with Cell-Titer-Glo reagent (Promega). To determine the IC₅₀ of cellular viability, cells were treated with MG132 or DBeQ at seven concentrations (threefold serial dilutions starting at 33 μM) for 48 h. IC₅₀ values were calculated from fitting the percentage of luminescence signal normalized to DMSO treated cells). Table V shows the IC₅₀ (μM) of ATPase activity of compounds described herein. FIG. 1A-B show the efficacy of compounds described herein against p97 mutations in vitro and in cancer cells. See also the methods described in Chou, et al., Proc. Natl. Acad. Sci. USA 2011, 108, 4834-4839. Compound 119 was determined, using the Caspase 3/7 glo assay described in Chou PNAS 2011, supra, to demonstrate the anticancer activity of Compound 119 is through induction of apoptosis.

TABLE V
IC₅₀ (μM) of ATPase activity

| | 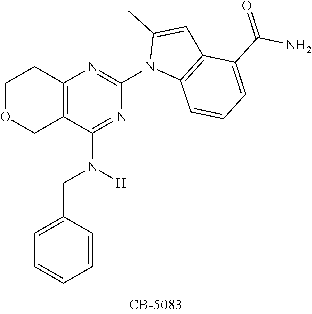 [SMILES: Cc1cc2c(C(N)=O)cccc2n1-c1nc2c(c(NCc3ccccc3)n1)COCC2] | 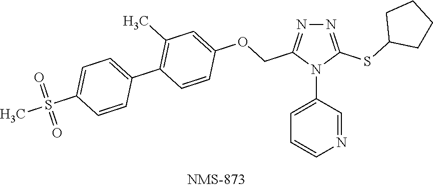 [SMILES: Cc1cc(OCc2nnc(SC3CCCC3)n2-c2cccnc2)ccc1-c1ccc(S(C)(=O)=O)cc1] | Compound 119 |
|---|---|---|---|
| WT | 0.013 | 0.038 | 0.018 |
| A530T | 0.010 | 0.138 | 0.044 |
| Q473P | 0.012 | 0.039 | 0.015 |
| N660K | 0.387 | 0.059 | 0.033 |
| P472L | 0.345 | 1.283 | 0.246 |
| T688A | 9.593 | 0.075 | 0.031 |
| E470K | | 0.112 | |
| E470D | | 0.110 | |
| E470Q | | 0.123 | |

ATPase assay procedure disclosed in Zhang, et al., Proc. Natl. Acad. Sci. USA, 112(14) E1705-E1714 (2015).

Table VI, below, shows the anti-cancer activity of NMS-873 (an allosteric and specific p97 inhibitor).

TABLE VI

| | | Anti-proliferative |
|---|---|---|
| | | activity |
| Cancer type | Cell line Name | (μM) of NMS-873 |
| Breast | MDA-MB-231 | 2.95 |
| Breast | MDA-MB-468 | 1.36 |
| Breast | MCF-7 | 3.07 |
| Colon | HCT15 | 2.1 |
| Colon | HCT116 | 1.2 |
| Colon | SW620 | 2.6 |
| blood cancer | Molt4 | 0.92 |
| blood cancer | CCRF-CEM | 0.10 |
| Multiple Myeloma | RPMI8226 | 0.08 |
| skin cancer | A375 (BRAFV600E) | 2.27 |
| skin cancer | SKMel2 (NRASQ61R) | 3.04 |
| skin cancer | SKMel5 (BRAFV600E) | 1.95 |
| skin cancer | LoxIMVI (BRAFV600E) | 0.65 |
| skin cancer | WM3918 (BRAF/NRAS WT) | 2.35 |
| Pancreatic cancers | PANC-1 | 5.00 |
| Pancreatic cancers | BxPC-3 | 2.50 |
| Lung Cancer | A549 | 2.20 |

TABLE VII
Anti-cancer activity of NMS-873 analogues

| | | Anti-proliferative activity (uM) | | |
|---|---|---|---|---|
| Structure | Compound | HCT116 | RPMI8226 | MM1S |
| 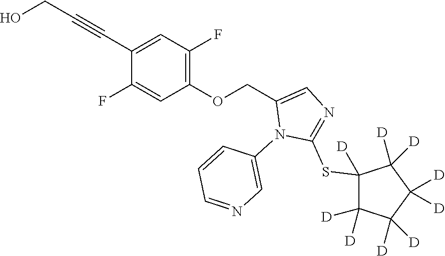 [SMILES: [2H]C1([2H])C([2H])([2H])C([2H])([2H])C([2H])(Sc2ncc(COc3cc(F)c(C#CCO)cc3F)n2-c2cccnc2)C1([2H])[2H]] | 123 | >42.11 | 24 | 16 |
| 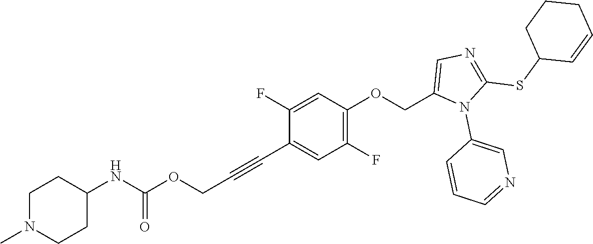 [SMILES: CN1CCC(NC(=O)OCC#Cc2cc(F)c(OCc3cnc(SC4C=CCCC4)n3-c3cccnc3)cc2F)CC1] | 119 | 7.63 | 1.87 | 1.43 |

* * *

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method of treating a multisystem degenerative disease or a neurodegenerative disorder, comprising administering to a patient in need of a compound of Formula (I): 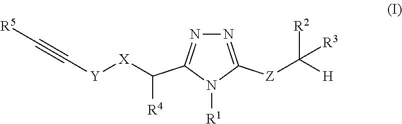
or a pharmaceutically acceptable salt, solvate or prodrug thereof, wherein:
Z is O, SO₀₋₂, NR, C(R⁷)₂, alkenyl or alkynyl;
R² and R³ are independently an optionally substituted C₁₋₉ cyclic, C₃₋₉ heterocyclic, or halogen, or R² and R³ together form an optionally substituted C₃₋₉ cyclic or 3- to 9-membered heterocyclic ring;
R¹ is optionally substituted phenyl or an optionally substituted 5- or 6-membered heteroaryl;
R⁴ is H, C(R⁷)₂, aryl, or heteroaryl;
X is O or SO₀₋₂;
Y is an optionally substituted alkyl, alkenyl, aryl, heteroaryl, cycloalkyl, or heterocycle;
R⁵ is H, nitrile, an optionally substituted aryl, an optionally substituted heterocycle, optionally substituted C₁-C₆ alkyl, optionally substituted C₃-C₉ cycloalkyl, or 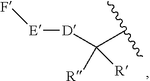
where
R′ and R″ are each independently selected from the group consisting of H, optionally substituted alkyl, —OR, halogen, —N(R)₂, and optionally substituted cycloalkyl or heterocycle;
or R′ and R″ may together form a 3- to 6-membered cycloalkyl or heterocycle that is optionally substituted;
D′ is selected from the group consisting of —O—, —NR—, —OCONR—, —NRSO₂—, —NRCO—, —NRSO₂NR—, —NRCOO—, —NRCONR—, and —NRC(NR)NR—;
E′ is selected from a bond or an optionally substituted C₁-C₆ alkyl and cycloalkyl; and
F′ is selected from the group consisting of H, an optionally substituted cycloalkyl, an optionally substituted heterocycle, an optionally substituted aryl, and an optionally substituted heteroaryl;
R is selected from the group consisting of H, optionally substituted alkyl, and optionally substituted cycloalkyl; and
R⁷ is selected from the group consisting of H, halogen, optionally substituted alkyl, optionally substituted cycloalkyl, —OR, and —N(R)₂.

2. The method of claim 1, wherein the multisystem degenerative disease is selected from the group consisting of inclusion body myopathy, Paget's disease of bone, frontotemporal dementia, and amyotrophic lateral sclerosis (ALS).

3. The method of claim 1, wherein the neurodegenerative disorder is selected from the group consisting of Alzheimer's disease (AD), Parkinson's disease (PD), Huntington's disease (HD), amyotrophic lateral sclerosis (ALS), and Batten disease.

4. A method of treating a hematological malignancy, comprising administering to a patient in need of a compound of Formula (I): 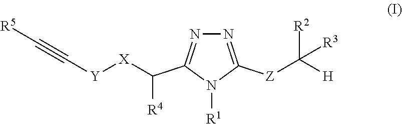
or a pharmaceutically acceptable salt, solvate or prodrug thereof, wherein:
Z is O, SO₀₋₂, NR, C(R⁷)₂, alkenyl or alkynyl;
R² and R³ are independently an optionally substituted C₁₋₉ cyclic, C₃₋₉ heterocyclic, or halogen, or R² and R³ together form an optionally substituted C₃₋₉ cyclic or 3- to 9-membered heterocyclic ring;
R¹ is optionally substituted phenyl or an optionally substituted 5- or 6-membered heteroaryl;
R⁴ is H, C(R⁷)₂, aryl, or heteroaryl;
X is O or SO₀₋₂;
Y is an optionally substituted alkyl, alkenyl, aryl, heteroaryl, cycloalkyl, or heterocycle;
R⁵ is H, nitrile, an optionally substituted aryl, an optionally substituted heterocycle, optionally substituted C₁-C₆ alkyl, optionally substituted C₃-C₉ cycloalkyl, or 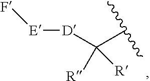
where
R′ and R″ are each independently selected from the group consisting of H, optionally substituted alkyl, —OR, halogen, —N(R)₂, and optionally substituted cycloalkyl or heterocycle;
or R′ and R″ may together form a 3- to 6-membered cycloalkyl or heterocycle that is optionally substituted;
D′ is selected from the group consisting of —O—, —NR—, —OCONR—, —NRSO₂—, —NRCO—, —NRSO₂NR—, —NRCOO—, —NRCONR—, and —NRC(NR)NR—;
E′ is selected from a bond or an optionally substituted C₁-C₆ alkyl and cycloalkyl; and
F′ is selected from the group consisting of H, an optionally substituted cycloalkyl, an optionally substituted heterocycle, an optionally substituted aryl, and an optionally substituted heteroaryl;
R is selected from the group consisting of H, optionally substituted alkyl, and optionally substituted cycloalkyl; and
R⁷ is selected from the group consisting of H, halogen, optionally substituted alkyl, optionally substituted cycloalkyl, —OR, and —N(R)₂.

5. The method of claim 4, wherein the hematological malignancy is leukemia, lymphoma or myeloma.

6. The method of claim 4, wherein the hematological malignancy is selected from the group consisting of acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL), Hodgkin's lymphoma, Non-Hodgkin's lymphoma, B-cell lymphoma, follicular lymphoma (FL), chronic lymphoid leukemia (CLL), diffuse large B-cell lymphoma (DLBCL), acute myeloid leukemia (AML), myeloproliferative diseases (MPD), and multiple myeloma (MM).

7. The method of claim 4, the hematological malignancy is multiple myeloma (MM).

8. The method of claim 4, wherein the hematological malignancy is relapsed and refractory.

9. A method of treating a cancer, comprising administering to a patient in need of a compound of Formula (I): 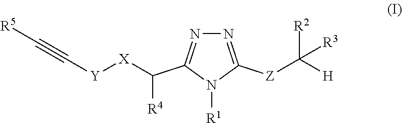
or a pharmaceutically acceptable salt, solvate or prodrug thereof, wherein:
Z is O, SO₀₋₂, NR, C(R⁷)₂, alkenyl or alkynyl;
R² and R³ are independently an optionally substituted C₁₋₉ cyclic, C₃₋₉ heterocyclic, or halogen, or R² and R³ together form an optionally substituted C₃₋₉ cyclic or 3- to 9-membered heterocyclic ring;
R¹ is optionally substituted phenyl or an optionally substituted 5- or 6-membered heteroaryl;
R⁴ is H, C(R⁷)₂, aryl, or heteroaryl;
X is O or SO₀₋₂;
Y is an optionally substituted alkyl, alkenyl, aryl, heteroaryl, cycloalkyl, or heterocycle;
R⁵ is H, nitrile, an optionally substituted aryl, an optionally substituted heterocycle, optionally substituted C₁-C₆ alkyl, optionally substituted C₃-C₉ cycloalkyl, or 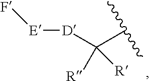
where
R′ and R″ are each independently selected from the group consisting of H, optionally substituted alkyl, —OR, halogen, —N(R)₂, and optionally substituted cycloalkyl or heterocycle;
or R′ and R″ may together form a 3- to 6-membered cycloalkyl or heterocycle that is optionally substituted;
D′ is selected from the group consisting of —O—, —NR—, —OCONR—, —NRSO₂—, —NRCO—, —NRSO₂NR—, —NRCOO—, —NRCONR—, and —NRC(NR)NR—;
E′ is selected from a bond or an optionally substituted C₁-C₆ alkyl and cycloalkyl; and
F′ is selected from the group consisting of H, an optionally substituted cycloalkyl, an optionally substituted heterocycle, an optionally substituted aryl, and an optionally substituted heteroaryl;
R is selected from the group consisting of H, optionally substituted alkyl, and optionally substituted cycloalkyl; and
R⁷ is selected from the group consisting of H, halogen, optionally substituted alkyl, optionally substituted cycloalkyl, —OR, and —N(R)₂.

10. The method of claim 9, wherein the cancer is selected from the group consisting of bladder cancer, liver cancer, colon cancer, rectal cancer, endometrial cancer, pancreatic cancer, small cell lung cancer, non-small cell lung cancer, breast cancer, urethral cancer, head and neck cancer, gastrointestinal cancer, stomach cancer, esophageal, ovarian cancer, renal cancer, melanoma, prostate cancer and thyroid cancer.

11. The method of claim 9, wherein the cancer is selected from the group consisting of breast cancer, colon cancer, blood cancer, multiple myeloma, skin cancer, pancreatic cancers, and lung cancer.

12. The method of claim 9, wherein the cancer comprises advanced solid tumor.

13. The method of claim 9, wherein the cancer is relapsed and refractory.

14. The method of claim 1, wherein the patient has a R155H mutation in the N domain of the p97 gene.

15. The method of claim 1, wherein the patient has a L198W mutation in the N-D1 linker of the p97 gene.

16. The method of claim 1, wherein the patient has a A232E mutation in the D1 domain of the p97 gene.

17. The method of claim 1, wherein the patient has a mutation in the p97 gene selected from the group consisting of K251A and E305Q.

18. The method of claim 1, wherein the patient has a mutation in the D2 domain of the p97 gene.

19. The method of claim 18, wherein the patient has a mutation in the p97 gene selected from the group consisting of K524A and E578Q.

20. The method of claim 18, wherein the patient has a mutation caused by ATP competitive inhibitor in the p97 gene selected from the group consisting of E470K, E470D, E470Q,P472L, Q473P, A530T, N660K, or T688A.
